(19) United States
(12) Patent Application Publication
(10) Pub. No.: US 2007/0020193 A1
(43) Pub. Date: Jan. 25, 2007

(54) Dronabinol compositions and methods for using same

(76) Inventors: M. H. de Vries, Weesp (NL); Jodi Miller, Marietta, GA (US)

Correspondence Address: MAYER, BROWN, ROWE & MAW LLP, P.O. BOX 2828, CHICAGO, IL, 60690-2828, US

(21) Appl. No.: 11/361,463

(22) Filed: Feb. 24, 2006

Related U.S. Application Data
(60) Provisional application No. 60/656,670, filed on Feb. 25, 2005.

(51) Int. Cl. A61L 9/04; A61K 31/353

(52) U.S. Cl. 424/45; 514/454

(57) ABSTRACT

In various embodiments, the present invention provides pharmaceutical compositions comprising delta-9-THC and to methods of administering such compositions to a patient in need of delta-9-THC therapy.

This application claims priority to U.S. provisional Application Ser. No. 60/656,670 filed Feb. 25, 2005, the entire contents of which is hereby incorporated by reference herein.

### FIELD OF THE INVENTION

The present invention relates to pharmaceutical compositions comprising delta-9-tetrahydrocannabinol (“delta-9-THC” or “THC”), to methods of administering such compositions to a patient, and to methods of treating various diseases and disorders.

### BACKGROUND OF THE INVENTION

Natural cannabinoid compounds can be obtained from several sources, and are frequently obtained from Cannabis Sativa. Natural cannabinoids can be used as a therapeutic agent for the treatment of a variety of diseases. For an overview of natural cannabinoid compounds see: David T. Brown ed., Cannabis, Harwood Academic Publishers 1998 ISBN 90-5702-291-5. The primary active cannabinoid in cannabis, delta-9-THC, has received much attention for its psychoactive properties, but this compound also displays analgesic, anti-spasmodic, anti-convulsant, anti-tremor, anti-psychotic, anti-inflammatory, anti-emetic and appetite-stimulant properties. A synthetic version of delta-9-THC, dronabinol, has been developed for medicinal purposes and has been marketed in the U.S. and elsewhere as an oral formulation under the commercial name MARINOL®. MARINOL® has been approved for use in the treatment of nausea and vomiting following cancer chemotherapy, and for treatment of anorexia associated with weight loss in patients with HIV. Currently, delta-9-THC is administered as soft gelatin capsules in sesame oil.

Oral administration, however, results in poor bioavailability due to extensive first-pass metabolism that yields both active and inactive metabolites. As a result, only 10-20% of an orally administered dose reaches systemic circulation, and maximum concentrations may not be reached for several hours. In contrast, smoking cannabis may result in rapid systemic absorption of delta-9-THC, and pharmacodynamic effects may be observed within minutes. However, smoking cannabis has numerous detrimental effects, including exposure to numerous carcinogenic chemicals and high variability in dosing and effect due to differences in the amount of active compound in the raw cannabis. Accordingly, there exists a great need for a convenient and safe method of administering delta-9-THC for rapid absorption without the unfortunate side effects associated with smoking cannabis, or the delayed action of other dosage forms.

### SUMMARY OF THE INVENTION

In various embodiments, the present invention provides pharmaceutical compositions comprising delta-9-THC and to methods of administering such compositions to a patient in need of delta-9-THC therapy.

In one embodiment, compositions of the invention comprise delta-9-THC in solution or suspension in a liquid vehicle. In another embodiment, the liquid vehicle comprises one or more of an alcohol, for example a C₁₋₄ alcohol such as ethanol and a propellant. In yet another embodiment, the delta-9-THC is present in the liquid vehicle in concentration of about 0.1 mg/50 mcL to about 2.0 mg/50 mcL

In another embodiment, the present invention provides methods of administering compositions of the invention to a patient using a metered dose inhaler. In a related embodiment, upon such administration, the patient achieves a blood plasma concentration of delta-9-THC of about 20 ng/mL to about 70 ng/mL at any time within about 10 minutes of the initiation of administration.

These and other aspects of the present invention are describe more fully herein below.

### BRIEF DESCRIPTION OF THE DRAWINGS

FIG. 1 shows pharmacokinetic and pharmacodynamic patient assessment flow charts.

FIG. 2 is a continuation of the patient assessment flow charts of FIG. 1.

FIG. 3 is a Linear and Semi-Logarithmic Geometric Mean plot of THC mean plasma concentrations for Groups I-III.

FIG. 4 is a Linear and Semi-Logarithmic Geometric Mean plot of 11-OH-THC mean plasma concentrations for Groups I-III.

FIG. 5 is a Linear and Semi-Logarithmic Geometric Mean plot of THC-COOH mean plasma concentrations for Groups I-III.

FIG. 6 is a table of summary pharmacokinetic data for Groups I-III for THC.

FIG. 7 is a table of summary pharmacokinetic data for Groups I-III for 11-OH-THC.

FIG. 8 is a table of summary pharmacokinetic data for Groups I-III for THC-COOH.

FIG. 9 is a plot of statistical analyses of Cₘₐₓ and AUC versus dose for THC for Groups I-II.

FIG. 10 is a plot of statistical analyses of Cₘₐₓ and AUC versus dose for 11-OH-THC for Groups I-II.

FIG. 11a is a plot of mean baseline adjusted heart rate for Groups I.

FIG. 11b is a plot of mean baseline adjusted heart rate for Groups II-III.

FIG. 12a is a plot of placebo corrected heart rate.

FIG. 12b is a plot of placebo corrected diastolic blood pressure.

FIG. 12c is a plot of placebo corrected systolic blood pressure.

FIG. 13 is a table of summary of the conjunctiva congestion for Groups I-III.

FIGS. 14-36 show plots and comparisons of the various cognitive test parameters as indicated:

FIG. 14a shows comparisons of Active versus Placebo alertness.

FIG. 14b is a plot of change in self-rated alertness from baseline for Groups I-III.

FIG. 15a shows comparisons of Active v. Placebo for calmness.

FIG. 15b is a plot of change in self-rated calmness from baseline for Groups I-III.

FIG. 16 is a plot of change in simple reaction time from baseline for Groups I-III.

FIG. 17 is a plot of change in choice reaction time from baseline for Groups I-III.

FIG. 18 is a plot of change in choice reaction time—accuracy from baseline for Groups I-III.

FIG. 19 is a plot of change in digit vigilance—speed from baseline for Groups I-III.

FIG. 20 is a plot of change in digit vigilance—targets detected from baseline for Groups I-III.

FIG. 21 is a plot of change in numeric working memory sensitivity index from baseline for Groups I-III.

FIG. 22 is a plot of change in numeric working memory—speed from baseline for Groups I-III.

FIG. 23 is a plot of change in spatial working memory sensitivity index from baseline for Groups I-III.

FIG. 24 is a plot of change in spatial working memory—speed from baseline for Groups I-III.

FIG. 25 is a plot of change in immediate word recall from baseline for Groups I-III.

FIG. 26 is a plot of change in delayed word recall from baseline for Groups I-III.

FIG. 27 is a plot of change in word recognition sensitivity index from baseline for Groups I-III.

FIG. 28 is a plot of change in word recognition—speed from baseline for Groups I-III.

FIG. 29 is a plot of change in picture recognition sensitivity index from baseline for Groups I-III.

FIG. 30 is a plot of change in picture recognition speed from baseline for Groups I-III.

FIG. 31 is a plot of change in tracking—average distance from target from baseline for Groups I-III.

FIG. 32 is a plot of change in power of attention from baseline for Groups I-III.

FIG. 33 is a plot of change in continuity of attention from baseline for Groups I-III.

FIG. 34 is a plot of change in quality of working memory from baseline for Groups I-III.

FIG. 35 is a plot of change in quality of episodic secondary memory from baseline for Groups I-III.

FIG. 36 is a plot of change in speed of memory from baseline for Groups I-III.

FIG. 37 is a Linear and Semi-Logarithmic Geometric Mean plot of THC mean plasma concentrations for Groups I-II.

FIG. 38 is a Linear and Semi-Logarithmic Geometric Mean plot of 11-OH-THC mean plasma concentrations for Groups I-II.

FIG. 39 is a Linear and Semi-Logarithmic Geometric Mean plot of THC-COOH mean plasma concentrations for Groups I-II.

FIG. 40 is a plot of mean baseline adjusted heart rate for Groups I.

FIG. 41 is a plot of mean baseline adjusted heart rate for Groups II.

FIG. 42 is a plot of placebo corrected mean heart rate for Group I.

FIG. 43 is a plot of placebo corrected mean heart rate for Group II.

FIGS. 44-62 show plots and comparisons of various cognitive test parameters.

FIG. 44 is a plot of change in self-rated alertness from baseline for Groups I-II.

FIG. 45 is a plot of change in self-rated contentment from baseline for Groups I-II.

FIG. 46 is a plot of change in self-rated calmness from baseline for Groups I-II.

FIG. 47 is a plot of change in simple reaction time from baseline for Groups I-II.

FIG. 48 is a plot of change in choice reaction time from baseline for Groups I-II.

FIG. 49 is a plot of change in choice reaction time—accuracy from baseline for Groups I-II.

FIG. 50 is a plot of change in digit vigilance—targets detected from baseline for Groups I-II.

FIG. 51 is a plot of change in digit vigilance—speed from baseline for Groups I-II.

FIG. 52 is a plot of change in numeric working memory sensitivity index from baseline for Groups I-II.

FIG. 53 is a plot of change in numeric working memory—speed from baseline for Groups I-II.

FIG. 54 is a plot of change in spatial working memory sensitivity index from baseline for Groups I-II.

FIG. 55 is a plot of change in spatial working memory—speed from baseline for Groups I-II.

FIG. 56 is a plot of change in immediate word recall from baseline for Groups I-II.

FIG. 57 is a plot of change in delayed word recall from baseline for Groups I-II.

FIG. 58 is a plot of change in word recognition sensitivity index from baseline for Groups I-II.

FIG. 59 is a plot of change in word recognition—speed from baseline for Groups I-II.

FIG. 60 is a plot of change in picture recognition sensitivity index from baseline for Groups I-II.

FIG. 61 is a plot of change in picture recognition speed from baseline for Groups I-II.

FIG. 62 is a plot of change in tracking—average distance from target from baseline for Groups I-II.

FIG. 63 is a plot of placebo corrected QTcB interval (Bazett's and Fredericia's) for Groups I-II.

FIG. 64 is a plot of baseline corrected QTcB interval (Bazett's and Fredericia's) for Group I.

FIG. 65 is a plot of baseline corrected QTcB interval (Bazett's and Fredericia's) for Group II.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited.

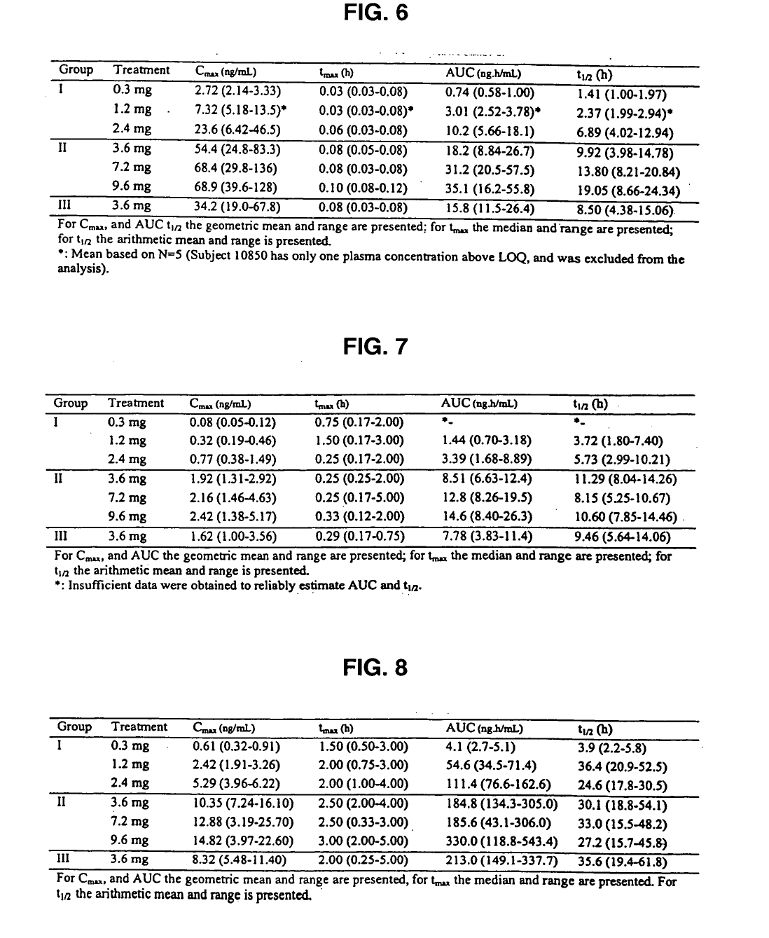

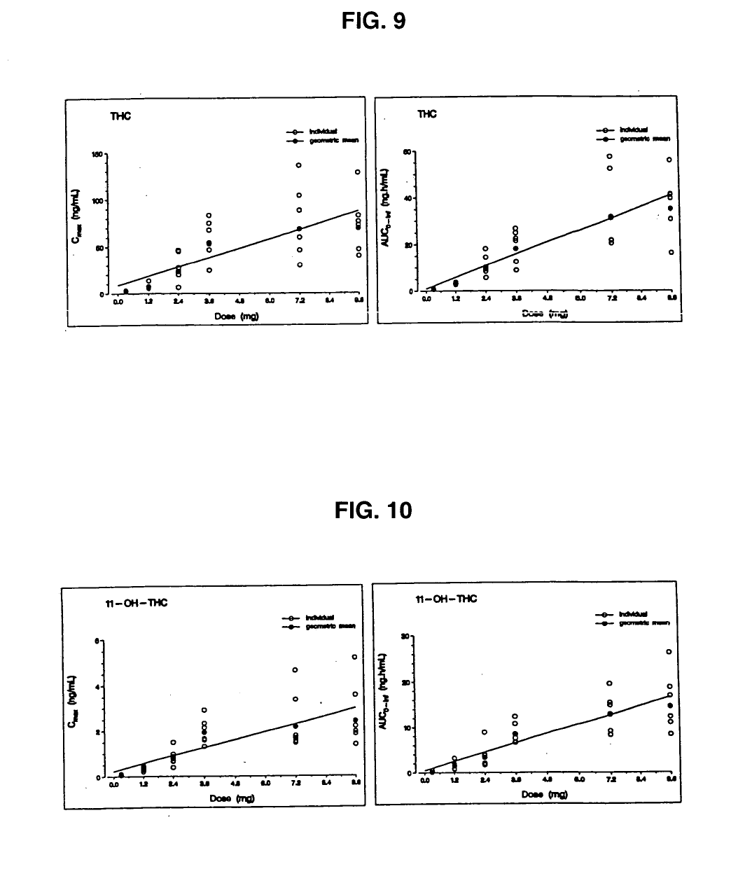

It is to be understood that any ranges, ratios and ranges of ratios that can be formed by any of the numbers or data present herein represent further embodiments of the present invention. This includes ranges that can be formed that do or do not include a finite upper and/or lower boundary. For example, by way of illustration and not limitation, referring to FIGS. 6 and 7, the ratio of the Cₘₐₓ values of THC to 11-OH-THC after 2.4 mg of delta-9-THC administered is 23.6 ng/ml:0.77 ng/ml, which is approximately 30:1. Accordingly, the skilled person will appreciate that such ratios, ranges and values are unambiguously derivable from the data presented herein.

As used herein, the terms “delta-9-THC” or “THC” or “delta-9-THC” are understood to refer to both natural and synthetic delta-9-tetrahydrocannabinol, and includes all salts, isomers, enantiomers, esters, prodrugs and derivatives of delta-9-THC.

In one embodiment, the present invention provides a metered dose inhaler comprising delta-9-THC wherein upon administration to a patient, therapeutically effective blood plasma levels of delta-9-THC are provided in a rapid manner. For example, in one embodiment, administration of delta-9-THC to a patient from a metered dose inhaler yields blood plasma concentrations of delta-9-THC of about 20 ng/mL to about 80 ng/mL in not more than about 15, about 14, about 13, about 12, about 11, about 10, about 9, about 8, about 7, about 6, about 5, about 4, about 3, about 2 or about 1 minutes from after initiation of administration.

In another embodiment, upon administration of a composition of the invention to a subject using a metered dose inhaler, therapeutically effective blood plasma concentrations of delta-9-THC are obtained. In various embodiments, blood plasma concentrations of at least about 5 ng/mL, at least about 10 ng/ml, at least about 20 ng/mL, at least about 25 mg/mL, at least about 30 ng/mL or at least about 40 ng/mL are obtained. In other embodiments, blood plasma concentrations of delta-9-THC of less than about 90 ng/mL, less than about 70 ng/mL, or less than about 50 ng/mL plasma are achieved upon administration of a composition of the invention to a subject using a metered dose inhaler. In another embodiment, blood plasma levels of delta-9-THC obtained in a patient by means of the present invention may be about 20 ng/mL plasma to about 70 ng/mL or about 30 ng/mL to about 60 ng/mL, or about 5 ng/mL to about 30 ng/mL, or about 10 ng/mL to about 20 ng/mL.

One embodiment of the present invention also provides for a rapid delivery of delta-9-THC to a patient by means of inhalation. For example, according to the methods of the present invention, peak blood plasma levels, such as those described above, may be obtained at any time within about 30 minutes after initiation of administration of the delta-9-THC dosage, such as within about 10 minutes, within about 5 minutes, or within about 2 minutes, or within 1 minute after initiation of administration of the delta-9-THC composition.

Compositions of the invention may be administered by a metered dose inhaler or by a portable, self-propelled inhalation administration system and may further comprise an optional adjuvant propellant, such as FDA-approved CFC's, propellants 11, 12, 114, 114A, hydrochlorofluorocarbons, hydrochlorocarbons, hydrocarbons, hydrocarbon ethers, compressed gases (e.g., nitrogen or carbon dioxide), propellants 152A, 142B, 22, R227, HFA-134A and mixtures of the forgoing. For example, the propellant may be 1,1,1,2-tetrafluoroethane (HFA-134a).

In one embodiment, compositions of the invention are administered using a non-ozone depleting pressurized metered dose inhaler. Such compositions may contain the pharmaceutically acceptable, non-ozone depleting hydrofluoroalkane propellants HFA 134a (1,1,1,2-tetrafluoroethane) and HFA 227 (1,1,1,2,3,3,3-heptafluoropropane), or a mixture thereof. In another embodiment, the present invention provides a non-ozone depleting pressurized metered dose inhaler comprising one or more doses of a composition of the invention.

Liquid Vehicle

In one embodiment, delta-9-THC is in solution in a liquid vehicle that is aerosolizable (capable of being aerosolized). The liquid vehicle may comprise delta-9-THC, one or more solvents or co-solvents and/or one or more propellants. A wide variety of solvents or co-solvents may be used in liquid vehicles suitable for the present invention, including, without limitation, low molecular weight branched and unbranched C₁-C₄ alcohols such as ethanol and propanol, and/or propylene glycol, glycerol or polyethylene glycol. Delta-9-THC may be present in the liquid vehicle in any suitable concentration, for example about 2% (w/w), or about 0.5% (w/w), or about 0.1 mg/50 mcL to about 2.0 mg/50 mcL, about 0.2 mg/50 mcL to about 1.5 mg/50 mcL, or about 0.8 mg/50 mcL to about 1.3 mg/50 mcL.

In one embodiment, where the liquid vehicle comprises a propellant that is a hydrofluoroalkane, the liquid vehicle may or may not contain a solvent such as ethanol. Higher percentages of solvent generally allow higher levels of dissolution of delta-9-THC.

In some embodiments, the liquid vehicle comprises about 100% propellant and about 0% solvent to about 85% propellant and about 15% solvent. In another embodiment, upon aerosolization of a composition of the invention using a metered dose inhaler, an aerosol spray is produced wherein at least about 5%, at least about 10%, at least about 15%, at least about 20%, or at least about 25% of the target dose (the dose intended to be administered) is in a fine particle mass with an aerodynamic particle size (by weight, volume or number) not greater than about 6 μm, not greater than about 5.9 μm, not greater than about 5.8 μm, not greater than about 5.7 μm, not greater than about 5.6 μm, or not greater than about 5.5 μm (Apparatus 1 (Anderson Cascade Impaction) described in U.S. Pat. No. <601>).

While the above liquid vehicle ratios reflect some embodiments of the invention, it will be recognized by those of skill in the art that the exact ratio of propellant to solvent in the liquid vehicle may vary according to the desired final concentration of delta-9-THC and droplet size. In one embodiment, any ratio of propellant to solvent that results in appropriate sized droplets and adequate dissolution of the delta-9-THC may be used in practice of this invention.

Those skilled in the art also will recognize that the “respirable dose” (or mass of delta-9-THC in particles with aerodynamic diameters small enough to be delivered to and absorbed by the lungs) may be increased by choosing Metered Dose Inhaler spray nozzles of various design and/or having smaller orifice diameters. Respirable doses may also be increased by extending the mouthpiece of the MDI in such a way as to create an integral or separate aerosol spacer or reservoir attached to the mouthpiece of the MDI. This promotes an increase in droplet evaporation and hence in the percentage of the active ingredient dose in smaller “respirable” particles or droplets. In one embodiment, a respirable droplet is less than 10 micrometers (μm) in diameter. The size of a droplet in an aerosol may be measured by cascade impaction and is characterized by the mass median aerodynamic diameter (MMAD) (the value for which 50% of the particles are larger or smaller). Using THC aerosols according to the present invention, an MMAD of about 2.5 μm or greater, or about 2.5 μm or smaller may be provided. In one aspect, the particle size distribution of the resulting aerosol (post actuator) may be determined using Anderson Cascade Impaction described in U.S. Pat. No. <601>. Sampling can occur at a flow rate of 28.3 liters of air per minute. The particle size distribution obtained from this test may be calculated on a per actuation basis. In some embodiments, at least about 20% of the target dose is in fine particle mass consisting of all drug with an aerodynamic particle size of less than about 5.8 μm.

In one embodiment, surface active agents or “surfactants” as valve lubricants and/or solubilizers are not required. This is in contrast to the invention of Purewal and Greenleaf (European Patent 0,372,777 (Riker Laboratories), Medicinal aerosol formulations) which provides HFA 134a/ethanol mixtures to produce stable formulations of pharmaceuticals in the presence of lipophilic surface active agents. Lipophilic surface active agents are incorporated in that invention in order to suspend undissolved material and to ensure adequate valve lubrication of the MDI. Without adequate valve lubrication, the useful life of the MDI and its ability to deliver an accurate dose of drug are severely attenuated. However, in one embodiment, compositions of the present invention do not require use of surface active agents.

Storage Stability

Delta-9-THC is known to deteriorate upon storage so that the effective concentration decreases and purity is vitiated. In one embodiment, compositions of the invention, upon storage in a closed container maintained at either room temperature, refrigerated (e.g. about 5-10° C.) temperature, or freezing temperature for a period of about 1, 2, 3, 4, 5, 6, 7, 8, 9, 10, 11, 12, 13, 14, 15, 16, 17, 18, 19, 20, 21, 22, 23, or 24 months, exhibits at least about 90%, at least about 92.5%, at least about 95%, at least about 97.5%, or at least about 99% of the original delta-9-THC present therein.

Delta-9-THC Dosing

In one embodiment, the dose of delta-9-THC received by a patient according to methods of the present invention may be, for example, about 1 to about 10 mg, about 2 mg to about 8 mg, or about 3 mg to about 4 mg per actuation of the inhaler. Such a delta-9-THC dose may be obtained from one to a small plurality (e.g. 1 to about 6) actuations of a metered dose inhaler. For example, it may be obtained from 2, 3, 4, 5, or 6 actuations. The doses described herein may be administered one to a small plurality of times per day, for example about 1, 2, 3, 4, 5 or 6 times per day.

Exemplary doses of delta-9-THC administered per actuation of the MDI or per inhalation include 0.1 mg to 50 mg per actuation, for example about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0 or 50 mg. In one embodiment, the MDI may deliver about 0.1 mg to about 10 mg delta-9-THC per actuation.

An MDI may contain multiple doses that may be delivered using multiple actuations. For example an MDI may be capable of delivering between about 1 and about 300 actuations, such as about 5, about 10, about 25, about 50, about 75, about 100, about 125, about 150, about 175, about 200, about 225, about 250, about 275 or about 300 actuations depending on the volume delivered per actuation.

In one aspect, an MDI may deliver about 25 to about 200 mcl of composition per actuation, for example, about 50 mcl, about 75 mcl, about 100 mcl, about 125 mcl, about 150 mcl, about 175 mcl or about 200 mcl. The choice of actuation volume is accomplished by evaluating a variety of parameters known to those of skill in the art, including mechanical aspects of selected nozzle, chemical and physical properties of the composition, acceptable delivery volumes, concentration of delta-9-THC desired or therapeutic dose and the like.

Pharmaceutical Excipients

Compositions of the invention optionally comprise one or more additional pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition.

Illustrative excipients include antioxidants, surfactants, adhesives, agents to adjust the pH and osmolarity, preservatives, thickening agents, colorants, buffering agents, bacteriostats, stabilizers, and penetration enhancers. Generally speaking, a given excipient, if present, will be present in an amount of about 0.001% to about 95%, about 0.01% to about 80%, about 0.02% to about 25%, or about 0.3% to about 10%, by weight.

Illustrative antioxidants for use in the present invention include, but are not limited to, butylated hydroxytoluene, butylated hydroxyanisole, potassium metabisulfite, and the like. One or more antioxidants, if desired, are typically present in a composition of the invention in an amount of about 0.01% to about 2.5%, for example about 0.01%, about 0.05%, about 0.1%, about 0.5%, about 1%, about 1.5%, about 1.75%, about 2%, about 2.25%, or about 2.5%, by weight.

In various embodiments, compositions of the invention comprise a preservative. Suitable preservatives include, but are not limited to, benzalkonium chloride, methyl, ethyl, propyl or butylparaben, benzyl alcohol, phenylethyl alcohol, benzethonium, or combination thereof. Typically, the optional preservative is present in an amount of about 0.01% to about 0.5% or about 0.01% to about 2.5%, by weight.

In one embodiment, compositions of the invention optionally comprise a buffering agent. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, or a magnesium buffering agent. Suitable buffering agents include carbonates, phosphates, bicarbonates, citrates, borates, acetates, phthalates, tartrates, succinates of any of the foregoing, for example sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasillicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthaiate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). Furthermore, combinations or mixtures of any two or more of the above mentioned buffering agents can be used in the pharmaceutical compositions described herein. One or more buffering agents, if desired, are present in compositions of the invention in an amount of about 0.01% to about 5% or about 0.01% to about 3%, by weight.

The foregoing excipients can have multiple roles as is known in the art. For example, some flavoring agents can serve as sweeteners as well as a flavoring agent. Therefore, classification of excipients above is not to be construed as limiting in any manner.

Treatment Methods

Compositions of the invention may be used to treat a variety of diseases and disorders including loss of appetite, anorexia, vomiting and nausea, for example, patients suffering from anorexia that is a symptom of AIDS or HIV infection, nausea and/or vomiting associated with cancer chemotherapy, pain, dementia, agitation, multiple sclerosis, and migraine headache. In such methods, a therapeutically effective amount of a composition of the invention is administered to the subject requiring treatment. Methods of treating and/or preventing these and other disorders by administering a composition of the invention to a subject in need thereof represent further embodiments of the present invention.

The related terms “therapeutically effective amount,” “prophylactically effective amount,” or “effective amount” as used herein refer to an amount of drug or agent that is sufficient to elicit the required or desired therapeutic and/or prophylactic response, as the particular treatment context may require.

Delta-9-THC administered by the methods of the present invention may also be used as an analgesic, anti-spasmodic, anti-convulsant, anti-tremor, anti-psychotic, anti-inflammatory, anti-emetic and appetite-stimulant. In one embodiment, a therapeutically effective amount of a composition of the invention is administered to a subject to treat suffering from migraines or multiple sclerosis.

These and many other aspects of the invention will be fully apparent by one of ordinary skill in the art in view of the examples set forth below. The examples provided herein are illustrative and are not to be construed as limiting the invention in any manner.

### EXAMPLES

### Example 1

A randomized, double blind, placebo controlled, three way crossover, single rising dose human clinical study in two sequential groups (Groups I and II) of nine young healthy male subjects (18-45 years of age and body mass index (BMI) of 20-26 kg/m²) of a metered dose inhalation composition according to one embodiment the present invention was conducted. Each subject received two ascending single doses of inhaled delta-9-THC and one single dose of inhaled placebo according to a three-way crossover balanced incomplete block design. The administered composition is detailed in Table 1. Composition I provides 0.3 mg per actuation, while Composition II provides 1.2 mg per actuation.

TABLE 1
Composition of delta-9-THC Metered Dose Inhaler

Quantity
(% w/w)
Composition

| Component | I | II |
|---|---|---|
| delta-9-THC | 0.5 | 2.0 |
| Ethanol (dehydrated alcohol) | 10 | 10 |
| Propellant HFA-134a | 89.5 | 88.0 |
| (1,1,1,2 tetrafluroethane) | | |

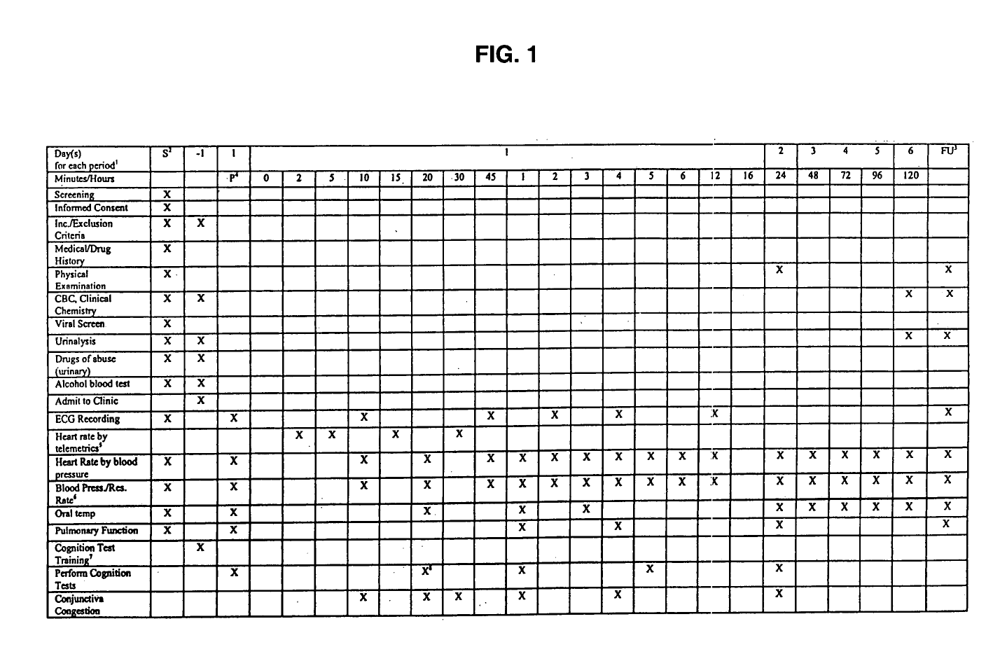

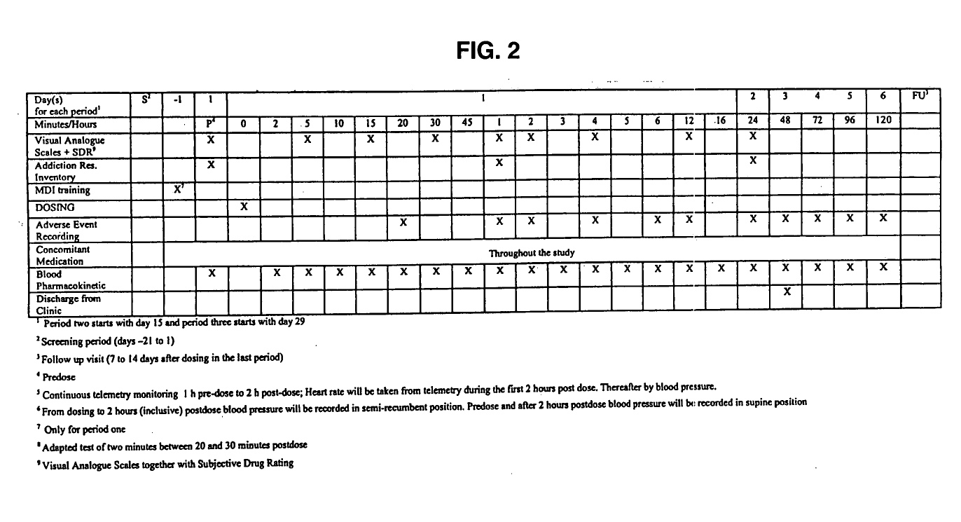

In each of three treatment periods, six subjects received delta-9-THC and three subjects received placebo. Each sequential dose level or period was separated by a washout period of two weeks, during which an interim safety and pharmacokinetic analysis was performed. A third group (Group III) of nine elderly healthy male and female subjects (65-80 years old and BMI 18-30 kg/m²) was added after completion of the sixth study period to participate in a single treatment period. Within Group III, six subjects received a single dose of delta-9-THC and three subjects received a single dose of placebo. Five dose levels were evaluated: 0.3 mg, 1.2 mg, and 2.4 mg in Group I, 3.6 mg, 7.2 mg, and 9.6 mg in Group II, and 3.6 mg in Group III. Subjects were confined to the study center from the day prior to dosing until the 48 hour blood sample. Subjects returned to the center for 72, 96 and 120 hour pharmacokinetic samples. Various pharmacokinetic and pharmacodynamic measurements and sampling were taken according to the Assessment Flow Charts in FIG. 1 and FIG. 2. Safety was measured by monitoring adverse events, physical examination, clinical laboratory and pulmonary function tests, vital signs, 12 lead ECG, and telemetry.

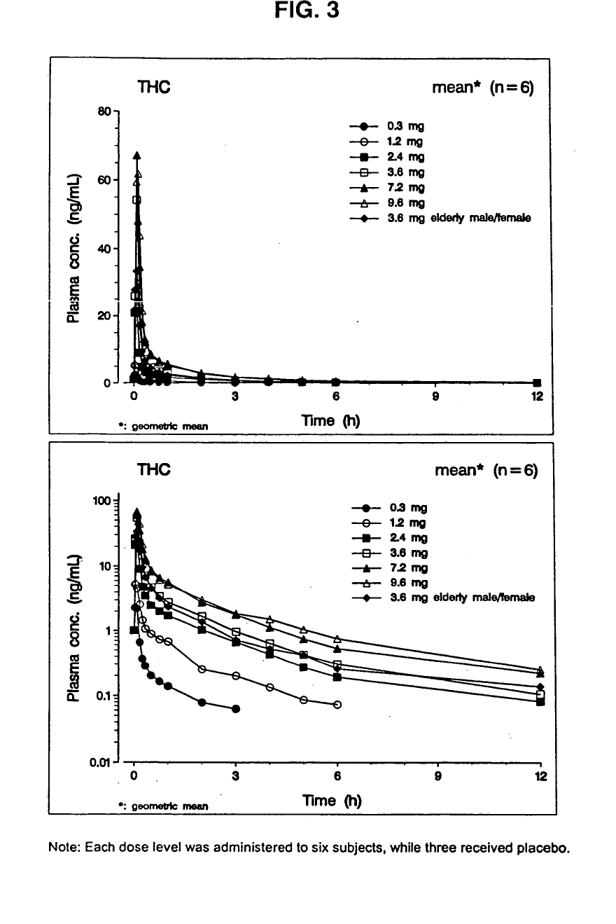

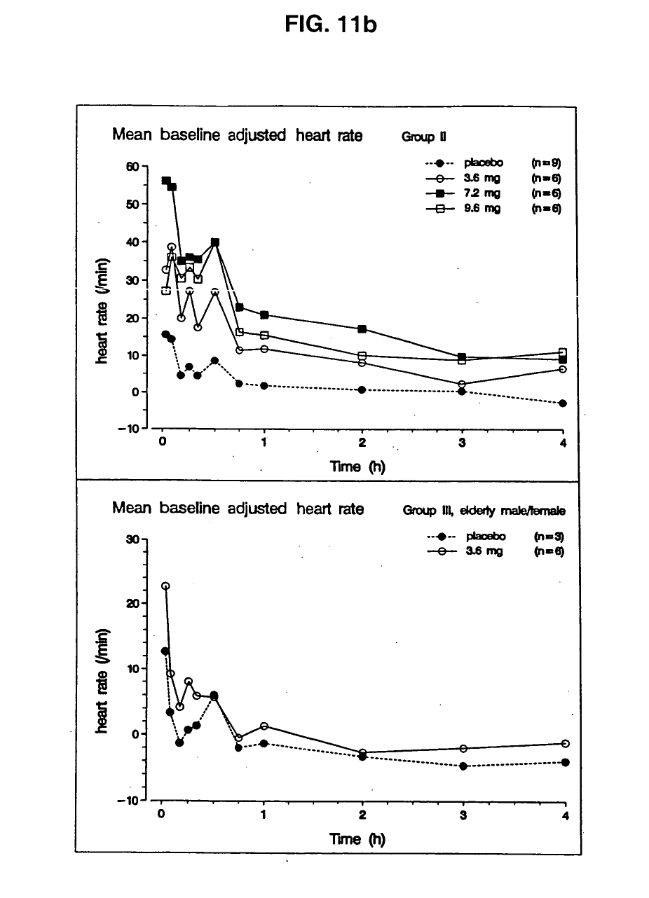

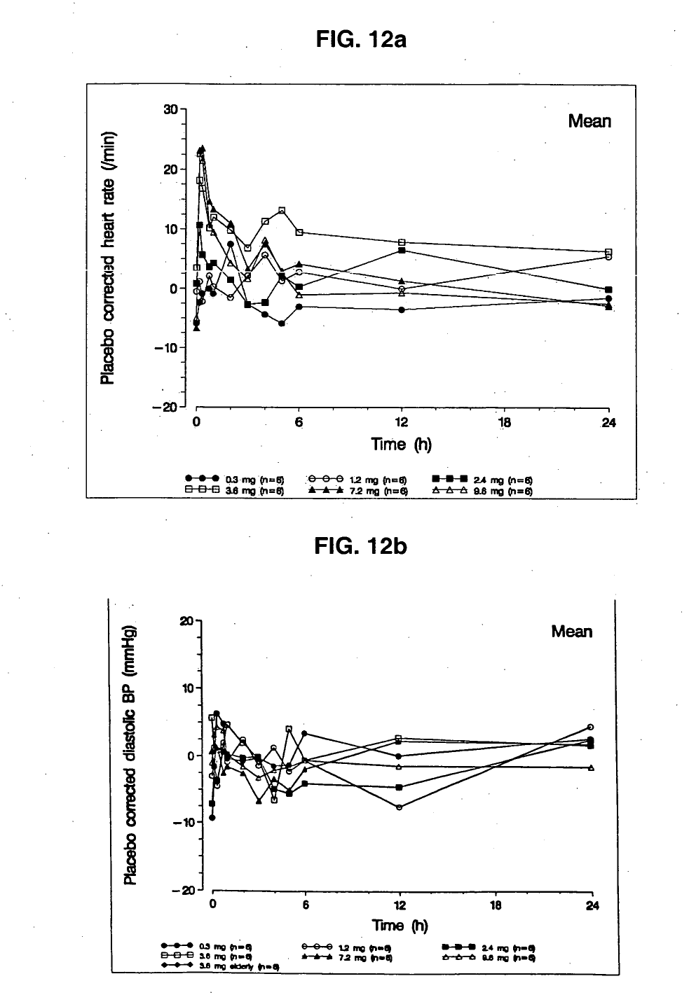

Safety, pharmacokinetic and pharmacodynamic variables were estimated. Plasma concentrations of delta-9-THC (THC), 11-hydroxy-tetrahydrocanabinol (11-OH-THC) and delta-9-tetrahydrocannbinol-carboxylic acid (THC-COOH) were determined for pharmacokinetic analysis. FIGS. 3, 4, and 5 show Linear and Semi-Logarithmic Geometric Mean THC, 11-OH-THC, and THC-COOH mean plasma concentrations obtained for various doses in Groups I-III. FIGS. 6-8 present summary pharmacokinetic data for Groups I-III for THC, 11-OH-THC and THC-COOH, respectively. FIGS. 9 and 10 show statistical analyses of Cₘₐₓ nd AUC versus dose for THC and 11-OH-THC, respectively.

Referring to FIGS. 3 and 6, plots of the linear and semi-logarithmic mean THC plasma concentration and the summary pharmacokinetic data indicate that the doses of the administered compositions achieved a Cₘₐₓ of about 2.7 ng/ml to about 70 ng/ml (depending on dose administered) in about 2 to about 6 minutes. Administration of a composition of the invention to a subject, and achievement of this result represent further embodiments of the invention.

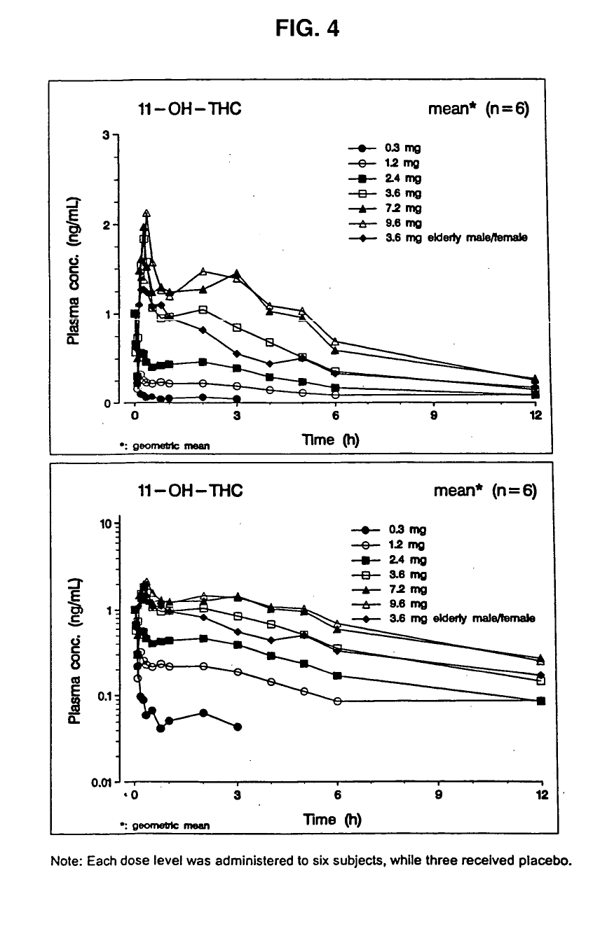

Referring to FIGS. 4 and 7, plots of the linear and semi-logarithmic 11-OH-THC plasma concentration and the summary pharmacokinetic data indicate that the doses of the administered compositions achieved a Cₘₐₓ from about 0.08 to about 2.5 ng/ml in from about 0.25 to about 1.5 hours. Administration of a composition of the invention to a subject, and achievement of this result represent further embodiments of the invention.

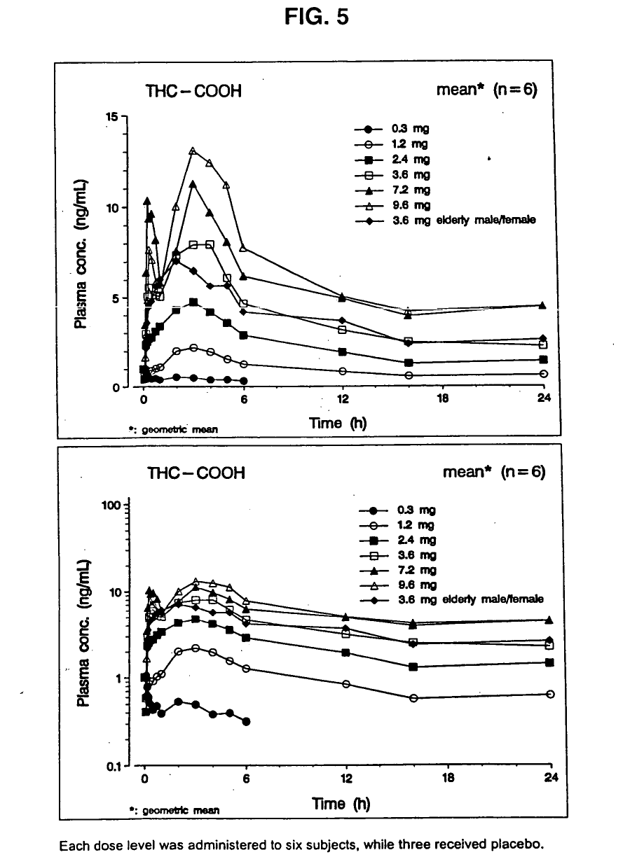

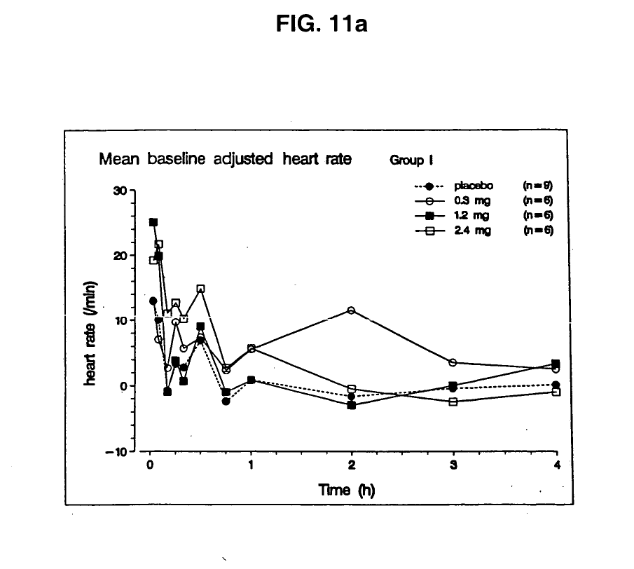

Referring to FIGS. 5 and 8, plots of the linear and semi-logarithmic THC-COOH plasma concentration and the summary pharmacokinetic data indicate that the doses of the administered compositions achieved a Cₘₐₓ from about 0.60 to about 15 ng/ml in from about 1.5 to about 3 hours. Administration of a composition of the invention to a subject, and achievement of this result represent further embodiments of the invention.

Referring to FIG. 9, a plot of statistical analyses of Cₘₐₓ and AUC versus dose for THC show a dose-related increase for both for Groups I and II for doses from 0.3-3.6 mg and a less than proportional increase for the 7.2 and 9.6 mg doses. Administration of a composition of the invention to a subject, and achievement of this result represent further embodiments of the invention.

Referring to FIG. 10, a plot of statistical analyses of Cₘₐₓ and AUC versus dose for 11-OH-THC show a dose-related increase for both for Groups I and II for doses from 0.3-3.6 mg and a less than proportional increase for the 7.2 and 9.6 mg doses.

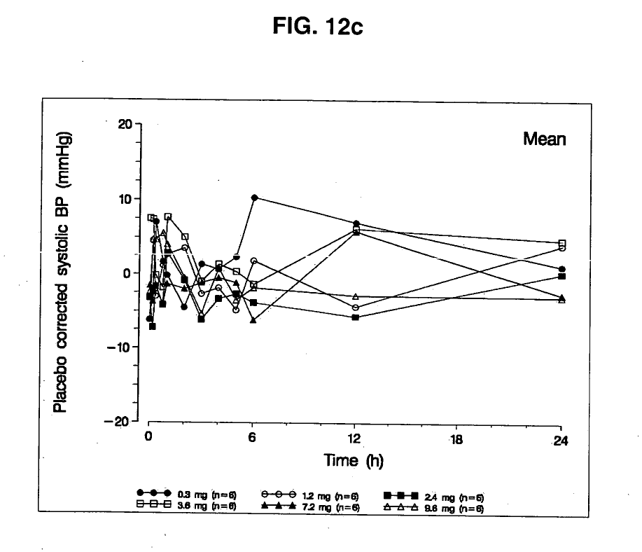

Referring to FIG. 11a-11b, a plot of mean baseline adjusted heart rate for Groups I-III show a dose-dependent increase in heart rate for Groups I-III. For Groups I-II, at the 0.3 mg dose, the effect on heart rate did not differ markedly from that observed with placebo, while at 1.2 mg doses an increase of approximately 12 beats per minute (bpm) relative to placebo was observed at 5 minutes after dosing and lasted for 5 minutes. At the higher doses (2.4, 3.6, 7.2 and 9.6 mg), a dose dependent increase in heart rate was observed lasting from about 15 minutes for 2.4 mg doses to about 2.0 hours for 9.6 mg doses. The largest effect on heart rate was observed at 7.2 mg doses and was about 46 bpm increase compared to placebo for doses from 0.3-3.6 mg and a less than proportional increase for the 7.2 and 9.6 mg doses. For Group III, the heart rate increased 10 bpm at 2 minutes and returned to normal 5 minutes after dosing. Administration of a composition of the invention to a subject, and achievement of this result represent further embodiments of the invention.

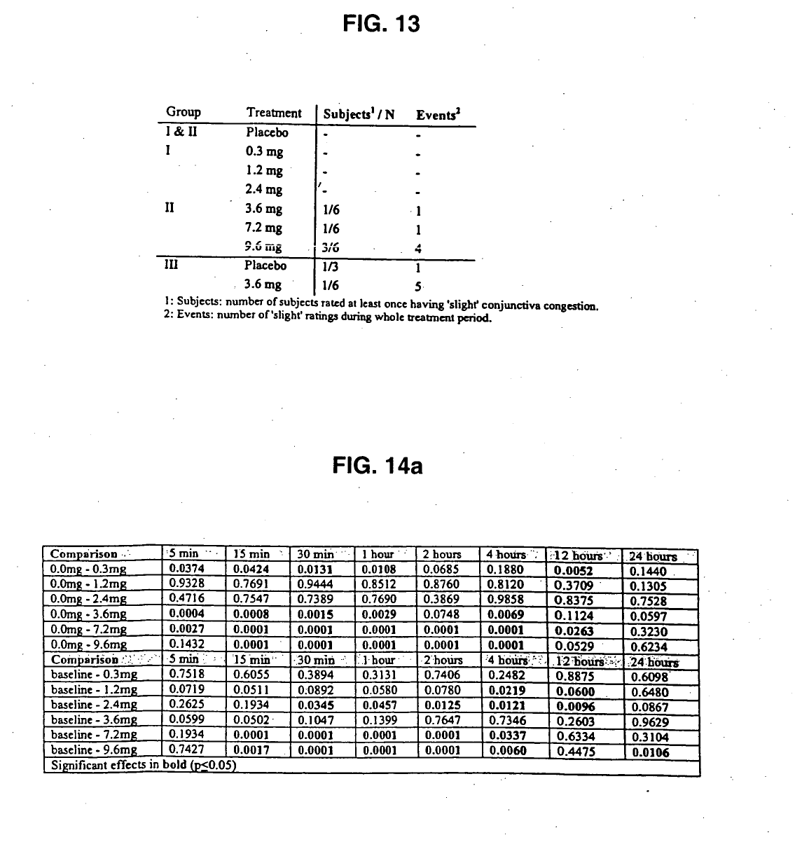

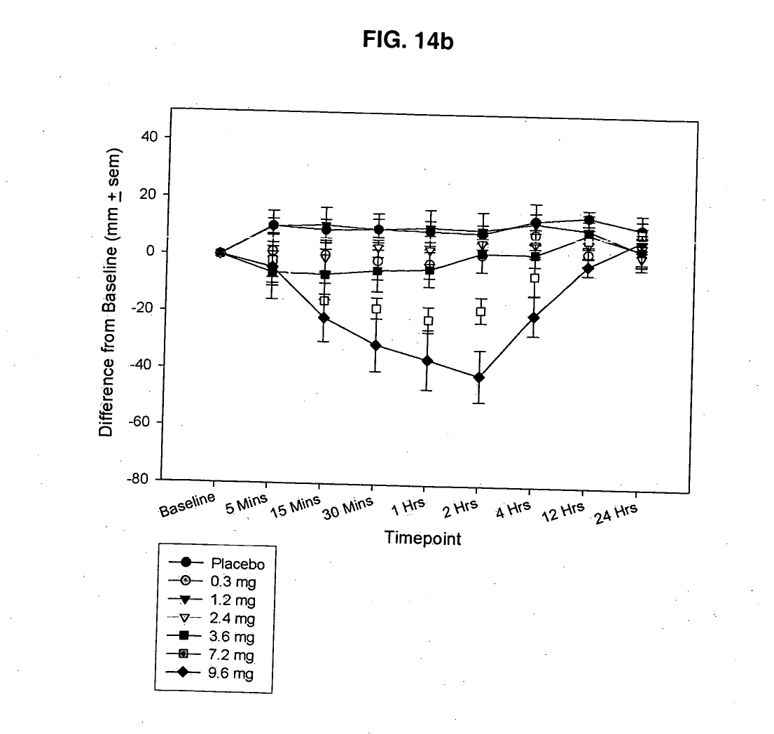

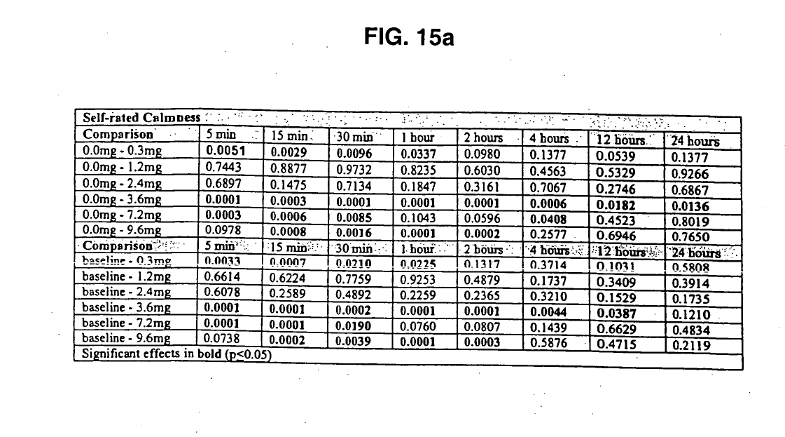

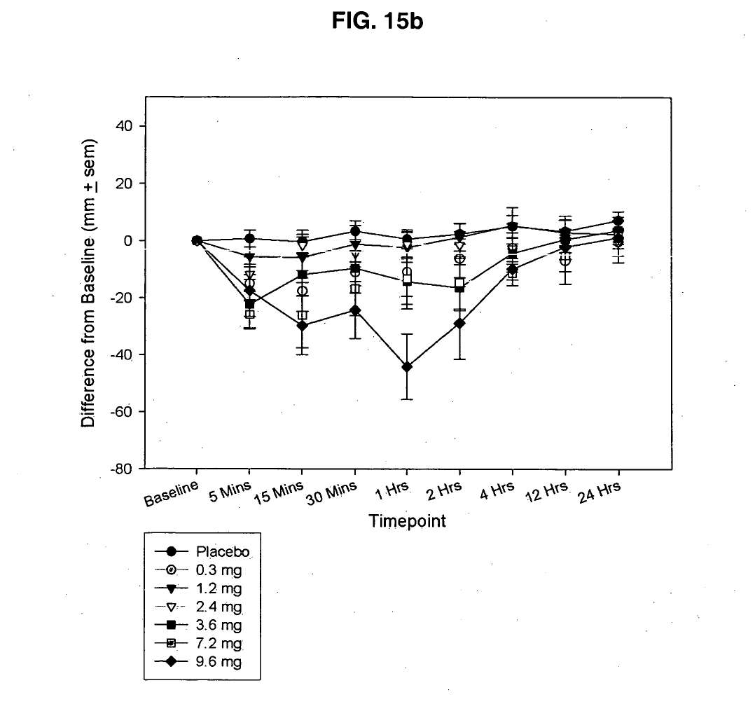

Heart rate, conjunctiva congestion, subjective ratings, and a battery of computerized cognitive tests were used to evaluate pharmacodynamics. FIG. 11a-b are plots of mean baseline adjusted heart rate for Groups I-III. FIG. 12a-c show plots of placebo corrected diastolic blood pressure. FIG. 13 shows a summary of the conjunctiva congestions for each group. FIG. 14a-b present comparisons of Active versus Placebo alertness and a plot of change in self-rated alertness. FIG. 15a-b show comparisons of Active v. Placebo for calmness and a plot of change in self-rated calmness.

Referring to FIG. 12a-c, plots of placebo corrected heart rate showed a dose related increase between 0.17 and 5 hours post dose. Mean placebo corrected heart rate showed a dose dependent effect in the first 3 hours post-dose with longer lasting effects in the 2.4 and 3.6 mg dose groups. Placebo corrected diastolic and placebo corrected systolic blood pressure indicate no clear dose-relationship across the dose levels investigated.

Referring to FIG. 13, a summary of the conjunctiva congestion for Groups I-III indicates non-clinically significant levels of conjunctiva congestion with apparent dose dependency. There was no or substantially no conjunctiva congestion for the 0.3 to 7.2 mg doses, with a slight conjunctiva congestion occurring once for each of the 3.6 and 7.2 mg doses, while the number of occurrences of slight congestion increased to 3 in the 9.6 mg dose group.

FIG. 14a shows comparisons of Active versus Placebo alertness.

FIG. 14b is a plot of change in self-rated alertness from baseline for Groups I-III.

FIG. 15a shows comparisons of Active v. Placebo for calmness.

FIG. 15b is a plot of change in self-rated calmness from baseline for Groups I-III.

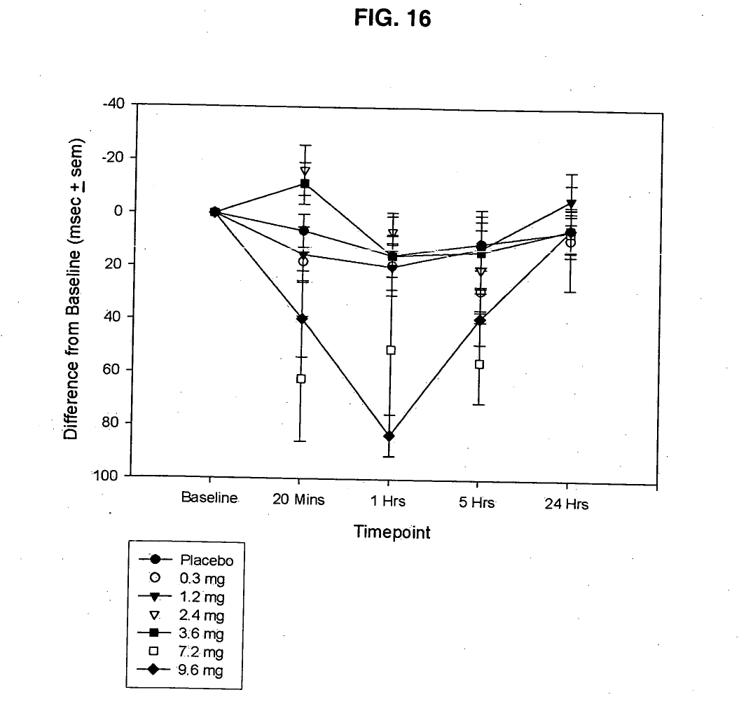

FIG. 16 is a plot of change in simple reaction time from baseline for Groups I-III.

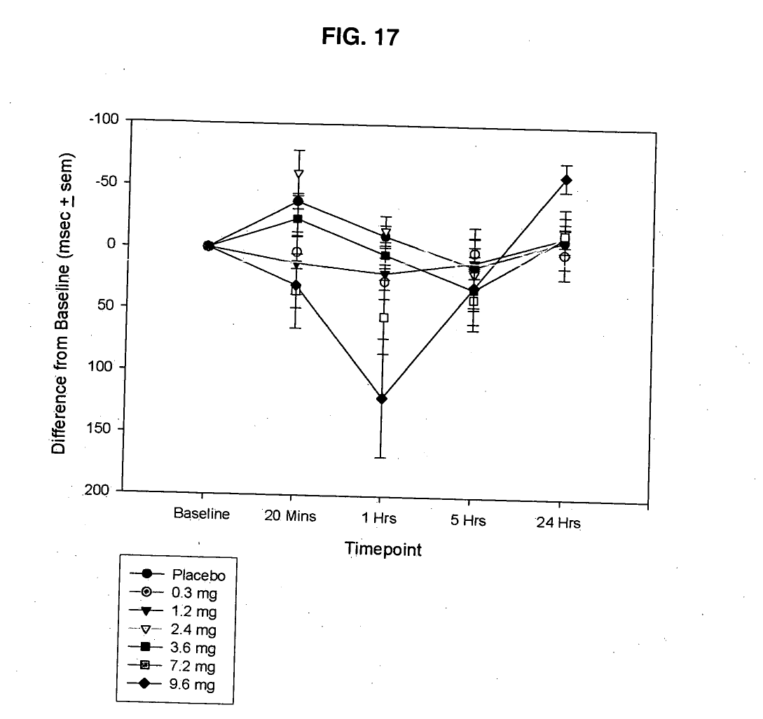

FIG. 17 is a plot of change in choice reaction time from baseline for Groups I-III.

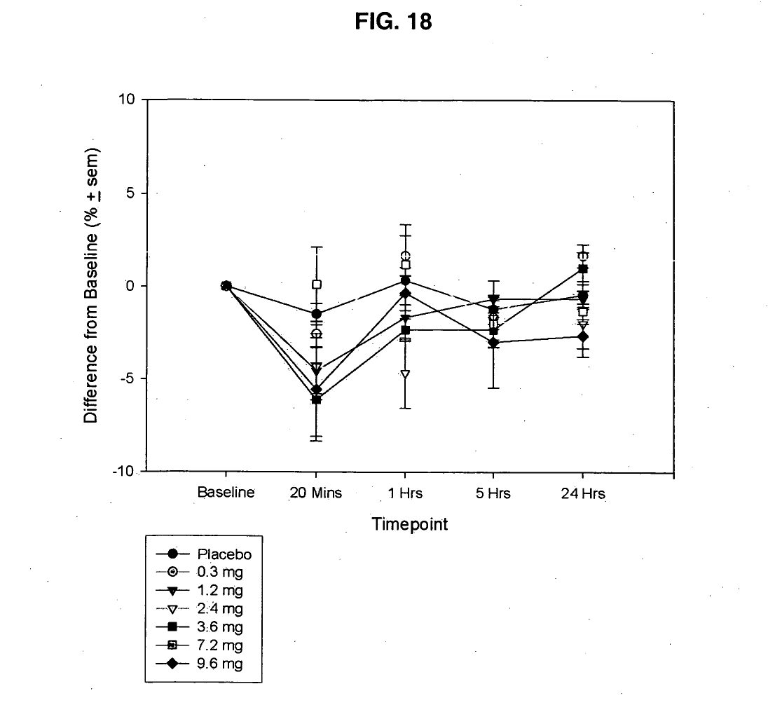

FIG. 18 is a plot of change in choice reaction time—accuracy from baseline for Groups I-III.

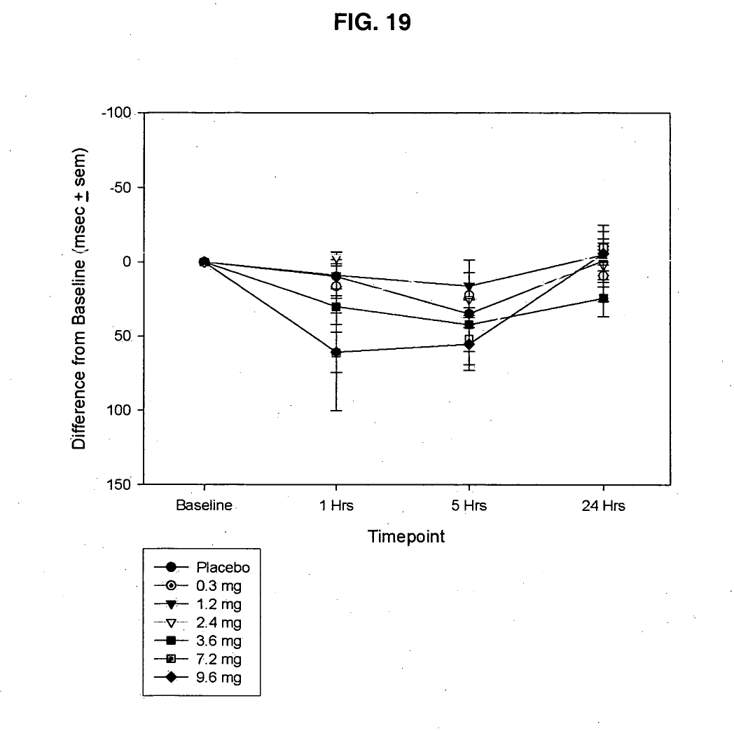

FIG. 19 is a plot of change in digit vigilance—speed from baseline for Groups I-III.

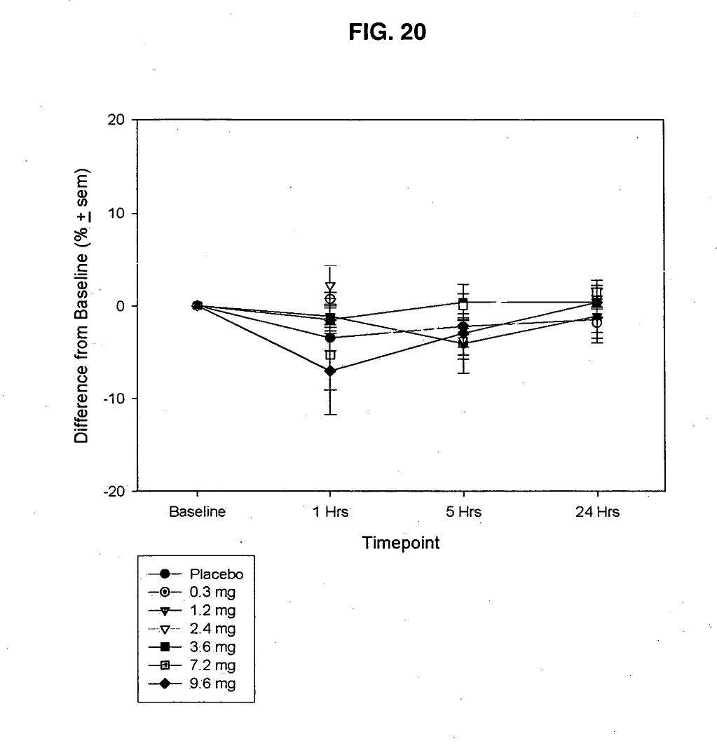

FIG. 20 is a plot of change in digit vigilance—targets detected from baseline for Groups I-III.

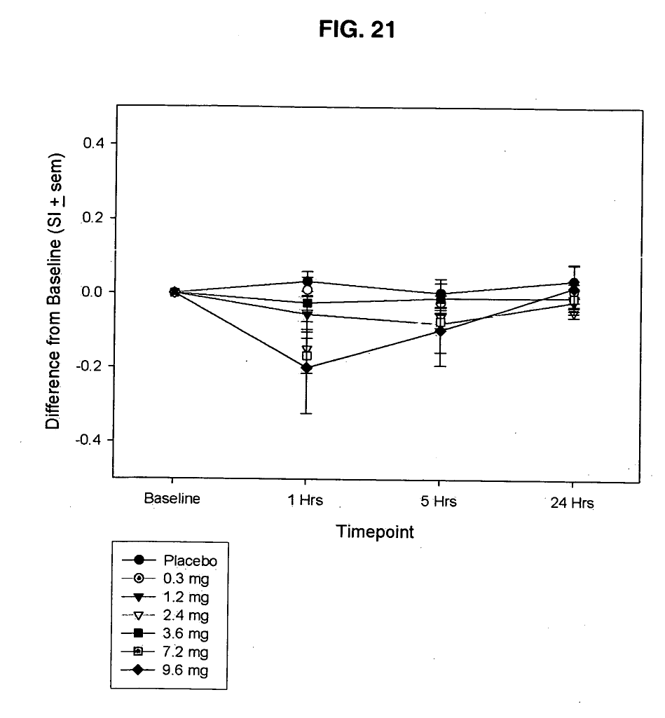

FIG. 21 is a plot of change in numeric working memory sensitivity index from baseline for Groups I-III.

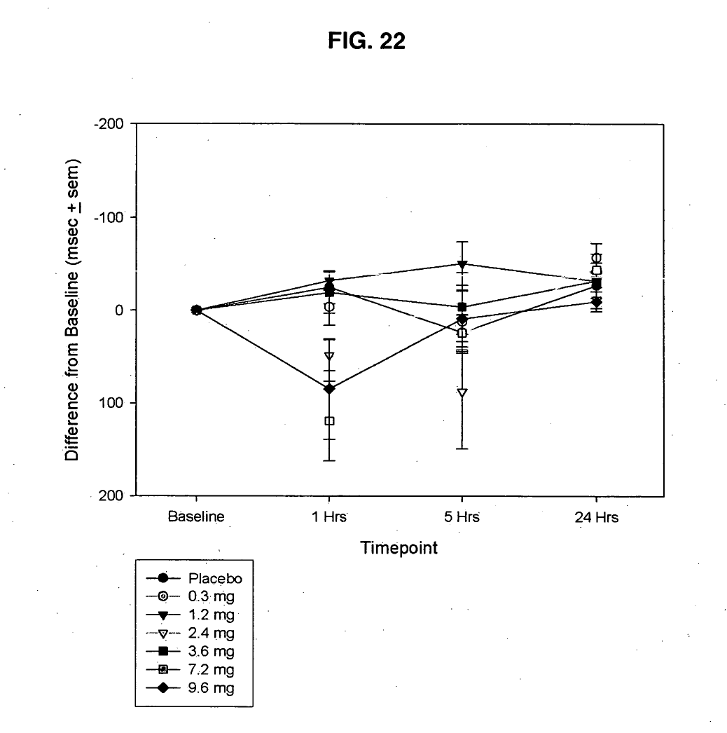

FIG. 22 is a plot of change in numeric working memory—speed from baseline for Groups I-III.

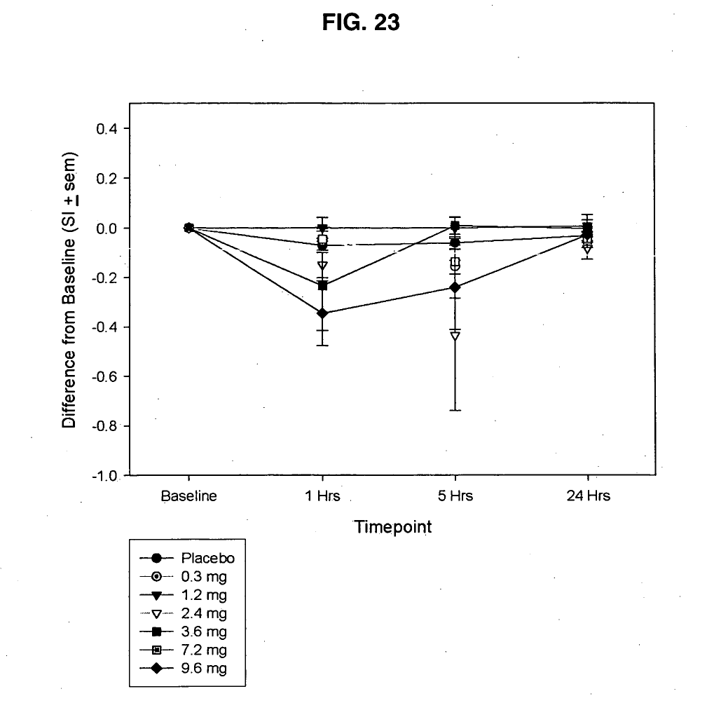

FIG. 23 is a plot of change in spatial working memory sensitivity index from baseline for Groups I-III.

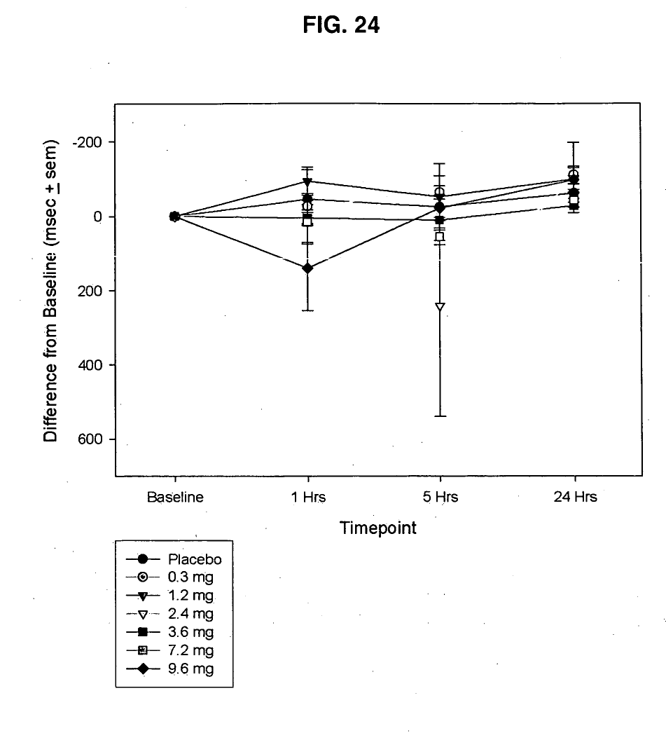

FIG. 24 is a plot of change in spatial working memory—speed from baseline for Groups I-III.

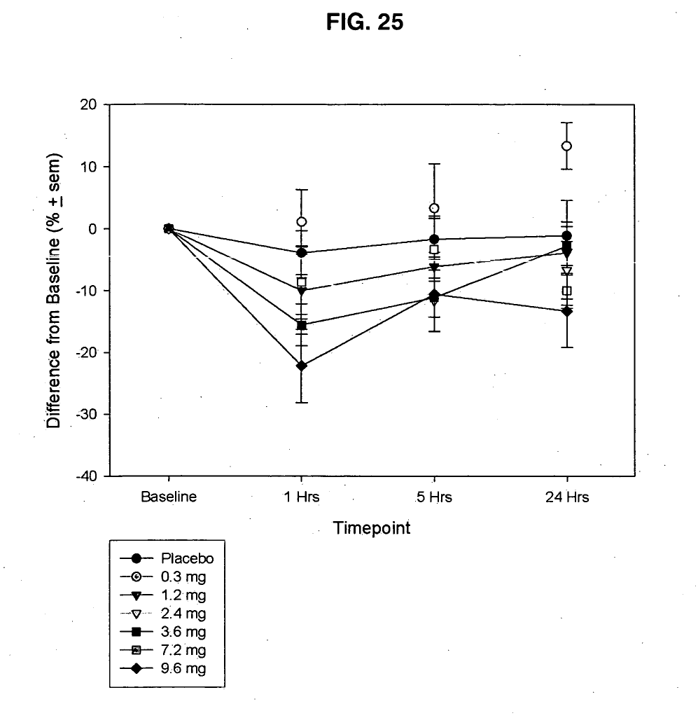

FIG. 25 is a plot of change in immediate word recall from baseline for Groups I-III.

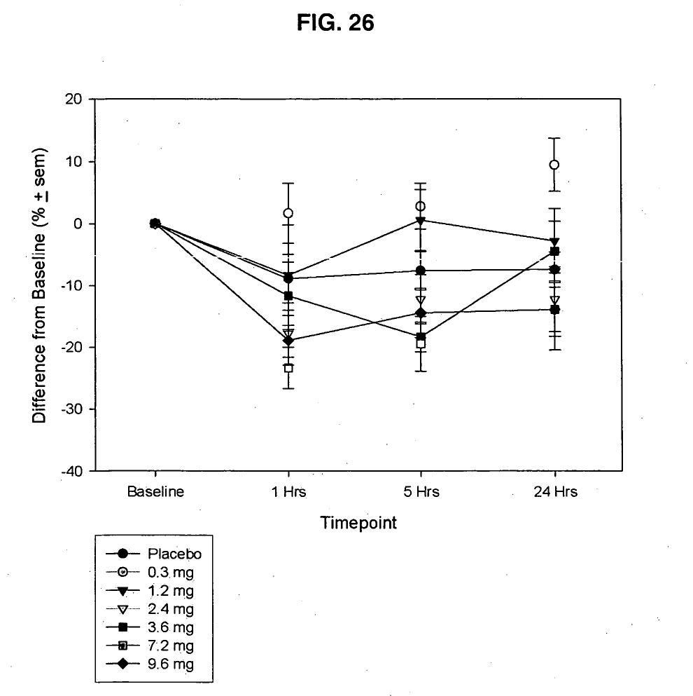

FIG. 26 is a plot of change in delayed word recall from baseline for Groups I-III.

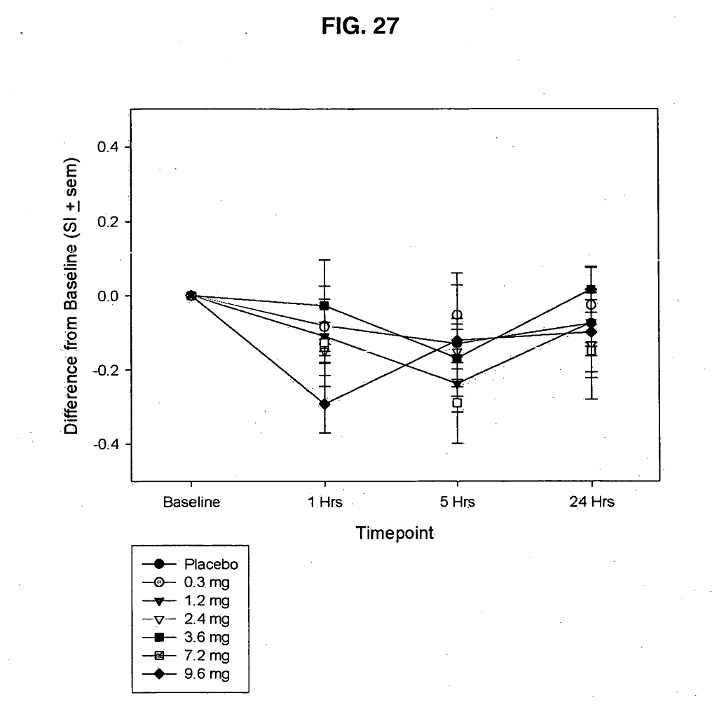

FIG. 27 is a plot of change in word recognition sensitivity index from baseline for Groups I-III.

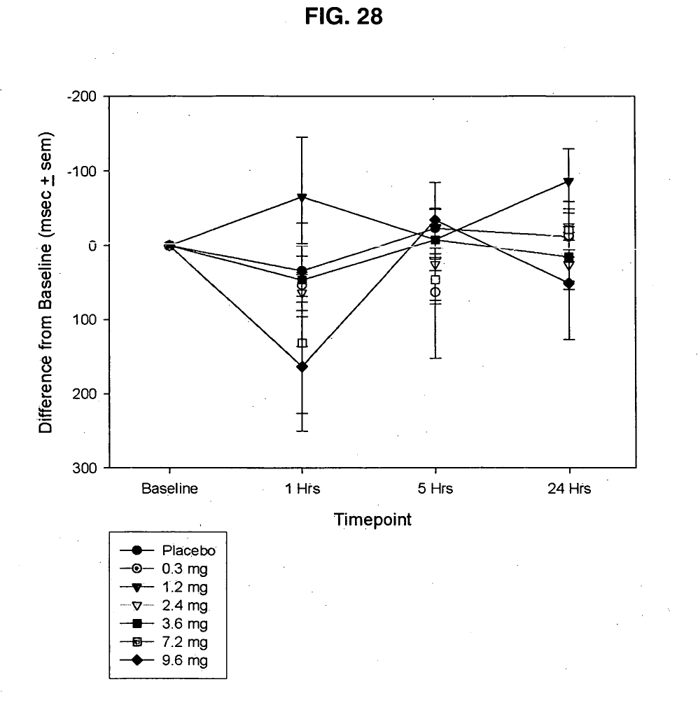

FIG. 28 is a plot of change in word recognition—speed from baseline for Groups I-III.

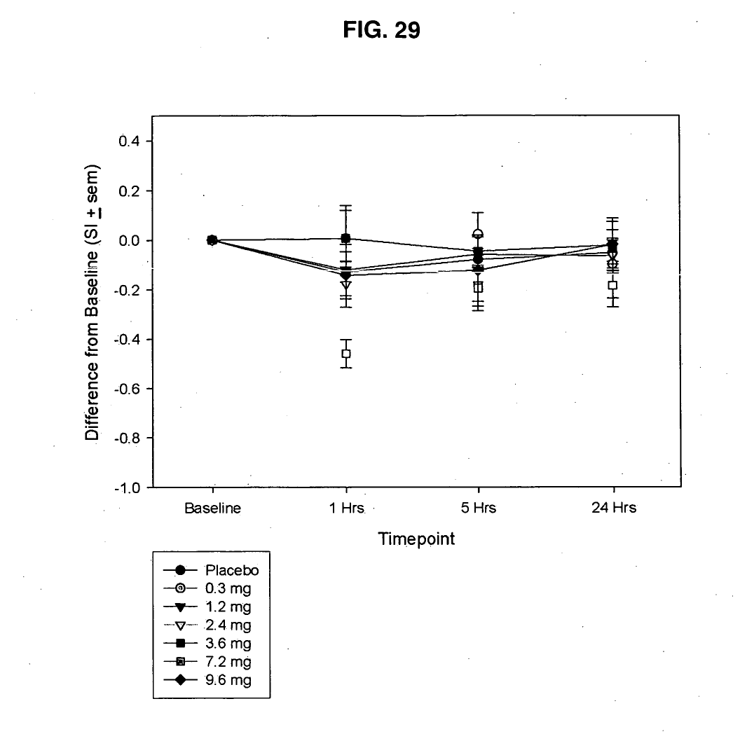

FIG. 29 is a plot of change in picture recognition sensitivity index from baseline for Groups I-III.

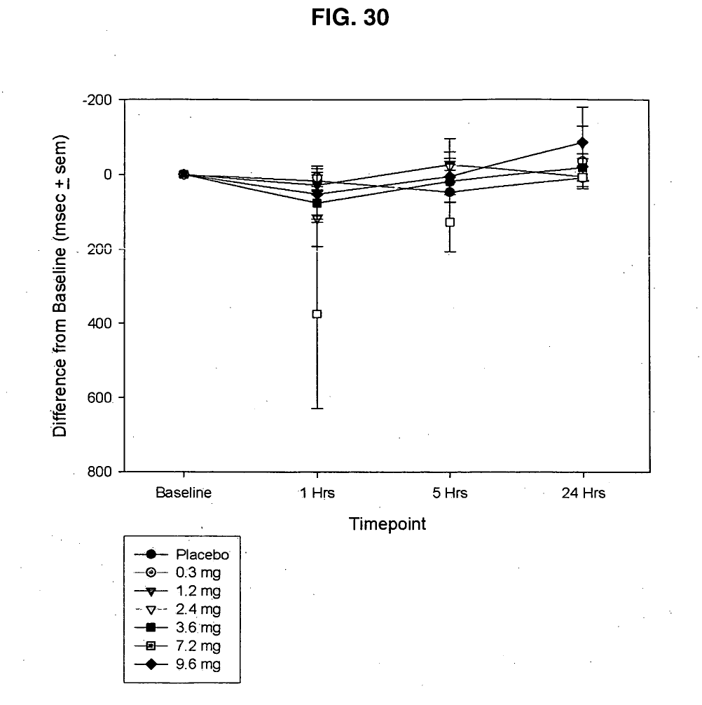

FIG. 30 is a plot of change in picture recognition speed from baseline for Groups I-III.

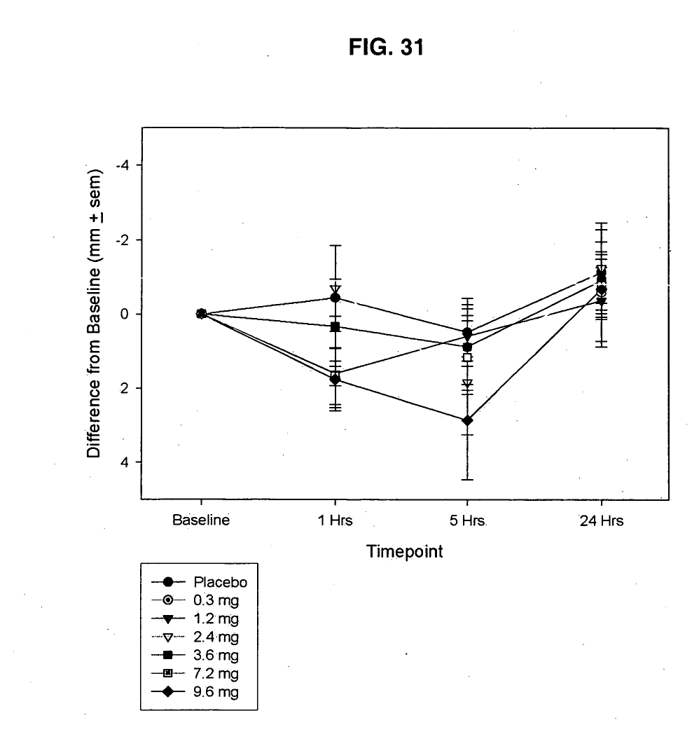

FIG. 31 is a plot of change in tracking—average distance from target from baseline for Groups I-III.

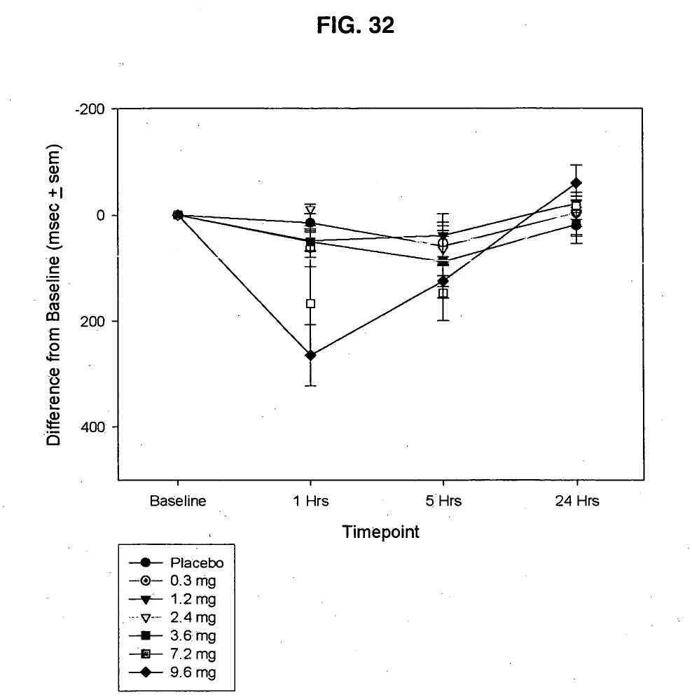

FIG. 32 is a plot of change in power of attention from baseline for Groups I-III.

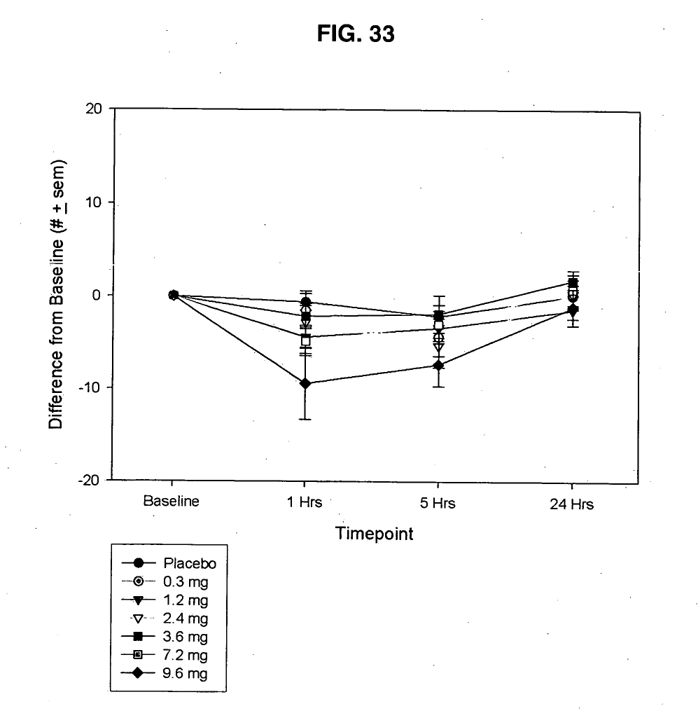

FIG. 33 is a plot of change in continuity of attention from baseline for Groups I-III.

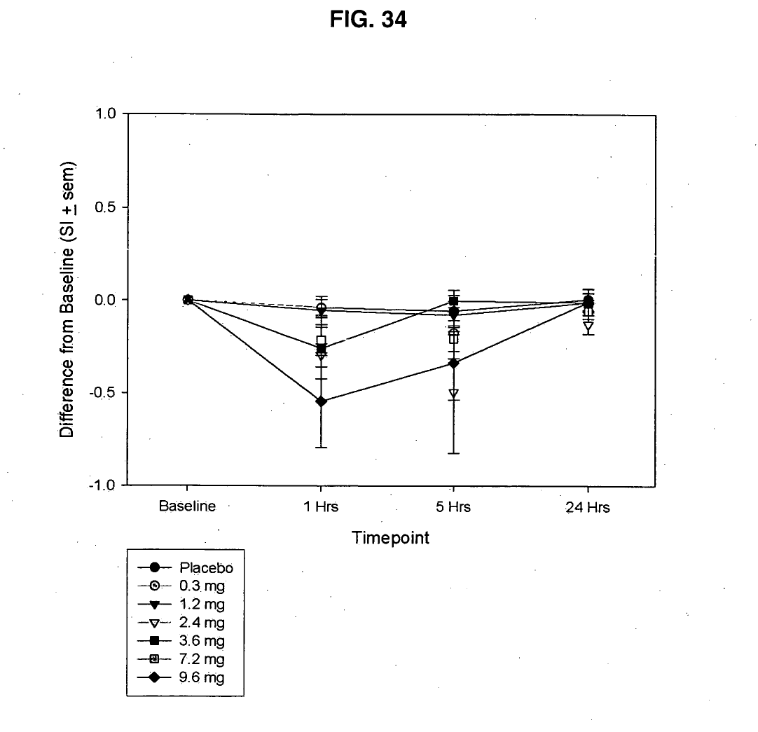

FIG. 34 is a plot of change in quality of working memory from baseline for Groups I-III.

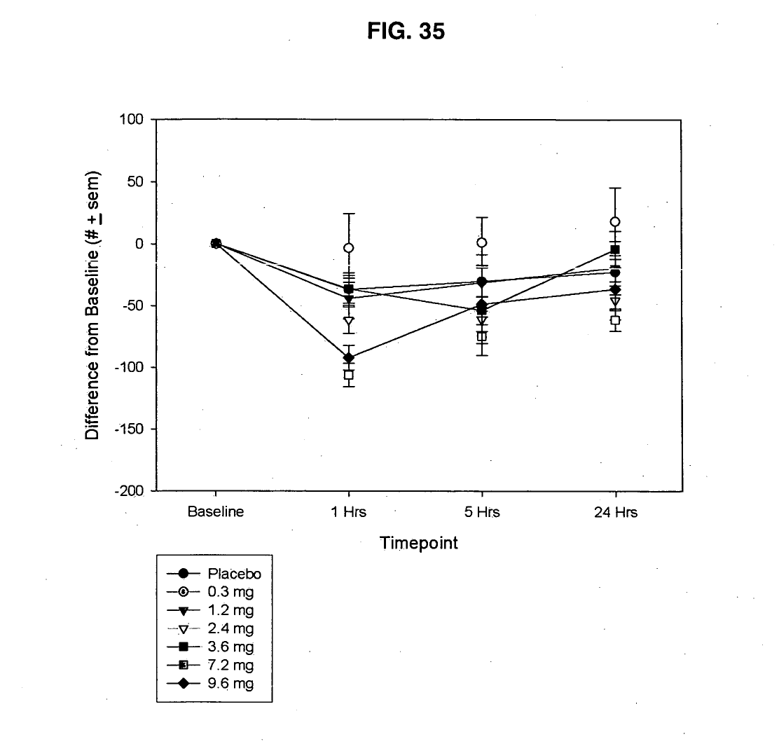

FIG. 35 is a plot of change in quality of episodic secondary memory from baseline for Groups I-III.

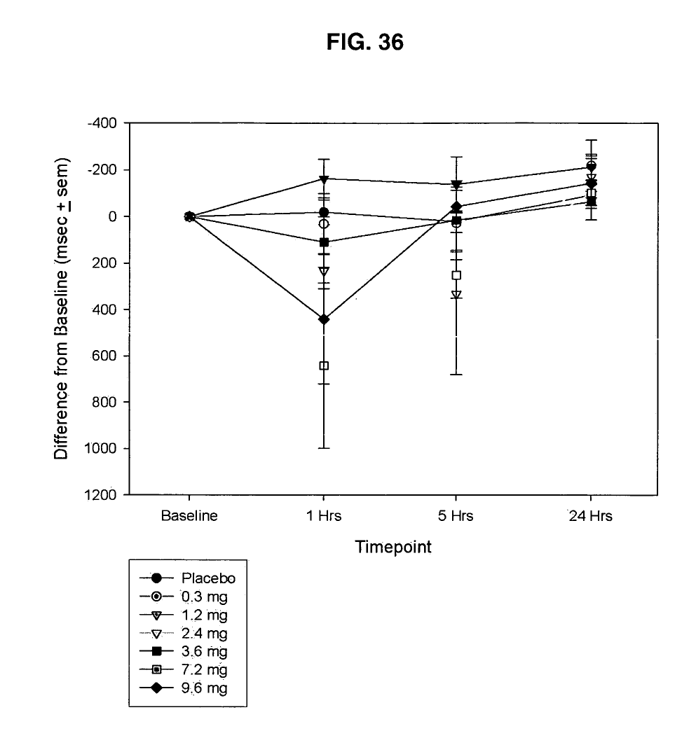

FIG. 36 is a plot of change in speed of memory from baseline for Groups I-III.

Simple Reaction Time: A benefit was seen for 3.6 and 2.4 mg doses at 20 minutes, while the 0.3 and 1.2 mg doses were generally equivalent to the placebo. The 7.2 mg and 9.6 mg showed a moderate decrement versus placebo with a peak decrement for 9.6 mg at 1 hour. Primary analysis indicated a mild early benefit for 2.4 mg, and decrements for 3.6, 7.2 and 9.6 mg. At 20 minutes significant decrements were seen for 7.2 mg (p<0.05) and 9.6 mg (p<0.05). At 1 hour a significant decrement was seen for 9.6 mg (p<0.05), and at 5 hours a significant decrement for 7.2 mg (p<0.05) was seen.

Choice reaction time: Small improvements at 20 minutes were seen with the placebo, with small decrements for 0.3 and 1.2 mg doses substantially equivalent to placebo, while the 2.4 and 3.6 mg doses were equivalent to placebo and 7.2 and 9.6 mg showed a moderate decrement. Primary analysis indicated significant decrements for 0.3 mg, 7.2 mg and 9.6 mg at 20 minutes (p<0.05). Significant decrements were also seen at 1 hour for 0.03 mg and 9.6 mg and at 24 hours for 9.6 mg.

Digit Vigilance: Little fluctuation from baseline was seen with placebo or the lower doses for the young subjects. A slight decrement was observed at 3.6 mg for young subjects at 1 hour, and more marked decrements for 7.2 and 9.6 mg a 1 hour. The elderly also showed a marked decrement at 3.6 mg at 1 hour. Primarily analysis failed to show significant dose-time interaction.

Numeric Working Memory: For the Numeric Working memory sensitivity index, flat profiles were observed for placebo and the lower doses in the young subjects with some indication of decline at 2.4, 7.2 and 9.6 mg at 1 hour with recovery thereafter. At 3.6 mg for the elderly, performance declined at 5 hours and further at 24 hours. No significant differences were seen in the analyses. For the Numeric Working Memory Speed, there was a flat profile with the placebo and low doses for the young, with indication of some decline at 7.2 and 9.6 mg at 1 hour and a decline at 2.4 mg at 5 hours. The elderly had a performance decline at 3.6 mg at 24 hours. A ‘speed-accuracy trade-off’ was also observed. At 1 hour significant decrements were seen at 7.2 (p<0.05) and 9.6 mg (p<0.05).

Spatial Working Memory: The Spatial Working Memory Sensitivity Index showed a flat profile with placebo and the active doses in the young subjects. There were indications of decline for the 9.6 mg dose at 1 hour, and the 2.4 mg dose at 5 hours. For the elderly, performance with placebo improved at 5 hours and declined for the 3.6 mg dose at 1 hour. No significant differences were observed from the analyses. For the Spatial Working Memory Speed, there was also a fairly flat profile in the active doses for the young with declines observed at 9.6 mg at 1 hour and 2.4 mg at 5 hours. For the elderly, decrements were observed at 1 hour for placebo and 3.6 mg, with recovery for placebo at 5 hours. No significant differences were observed.

Cognitive Episodic Secondary Memory Tasks: Immediate word recall showed a flat profile with placebo and some general declines with the active doses, though clear separation was observed for 3.6 mg at 1 hour and for 9.6 mg across the study. There was a slight indication of improved performance at 0.3 mg. For the elderly, the placebo showed a flat profile, while performance declined at 1 hour for 3.6 mg, with recovery at 24 hours. No significant differences were found. For delayed word recall, the data showed a flat profile for placebo and indicated declines with 2.4, 3.6, 7.2 and 9.6 mg and some slight improvement at 0.3 mg and at 5 hours for 1.2 mg. For the elderly, performance between placebo and 3.6 mg was generally equivalent with some improvement at 5 hours for placebo. The analysis indicated no significant dose-time interaction, but a significant main effect of dose was seen (p<0.05). The overall comparisons indicated support for benefits at 0.3 and 1.2 mg and decrements at 3.6, 7.2 and 9.6 mg. The word sensitivity index had a flat profile with placebo and indications of declines for the active doses, most notable for 9.6 mg at 1 hour and 7.2 mg at 5 hours. For the elderly performance at the 3.6 mg was slightly superior at the 3.6 mg dose compared to placebo. No significant differences were shown. The word recognition speed had a fairly flat profile with placebo and active doses for young subjects. There were indications of declines for 7.2 and 9.6 mg and improvement at 1.2 mg at 1 hour. For the elderly, performance with placebo improved at 1 hour and showed a marked decline with 3.6 mg at 1 hour. The analyses did not show any significant differences.

For the picture recognition sensitivity index, there was a flat profile with placebo and the active doses. A single marked decline for 7.2 mg at 1 hour was seen. For the elderly, performance improved at 1 hour, while a fairly flat profile was seen for 3.6 mg. The analyses did not show any significant differences. For picture recognition speed, there was a flat profile with the placebo and active, with a slight indication of decline at 7.2 mg at 1 hour. The elderly showed improved performance at 5 and 24 hours with placebo and little change with 3.6 mg. No significant differences were found.

For the tracking task, there was a fairly flat profile with placebo and active. Elderly performance improved at 1 and 24 hours for placebo with declines at the same timepoints for 3.6 mg.

The subjective drug ratings for Group I showed maximum psychoactivity (“feel the drug” scores) at 0.3 mg with corresponding highest mean “liking” scores. As dose increased, “disliking” increased to a maximum at 2.4 mg. For Group II, the maximum “liking” scores occurred at 3.6 mg, with maximum “disliking scores at 7.2 mg and maximum “feel the drug” scores at 7.2 mg. For Group III, there was a difference between placebo and 3.6 mg for each of the three scales.

ARCI showed no difference on baseline score, with significant differences at 1 hour post—dose for the Benzedrine Group and the PCAG scale. No significant results were seen for the MBG, LSD or the Amphetamine Scales. No differences were observed between the elderly and the young.

The effect on heart rate showed a maximum at 2-5 min after single dosing with a duration of approximately 2 hours. Both duration and maximum effect coincided with delta-9-THC, but not 11-OH-THC or THC-COOH maximum plasma concentrations. The delayed increase in 11-OH-THC plasma concentrations between 2 and 4 hours was not associated with a clear effect on heart rate. The effects on cognitive functioning and VAS showed a delay of up to one hour and up to two hours respectively.

### Example 2

A randomized, double-blind, placebo-controlled, multiple rising dose safety, tolerability, pharmacokinetic and pharmacodynamic study to assess two dose levels of inhaled delta-9-THC was conducted. Dose levels were studied in ascending order. Two consecutive groups (n=9/group) of healthy subjects were studied (Groups I and II). In each group, six subjects received active treatment and three subjects received placebo. There was a lapse of at least 10 days between the groups for interim safety and pharmacokinetic analysis. Two dose levels were studied in ascending order. Within each group, six subjects received active treatment and three subjects received placebo treatment. Subjects in each treatment Group (I or II) received single and multiple doses of inhaled delta-9-THC according to the following schedule:

Group I

Day 1: One dose of 1.2 mg delta-9-THC or placebo administered in the morning.

Days 5-12: Multiple dose administration (1.2 mg delta-9-THC or placebo three times daily—every 8 hours); first dose on Day 5 in the morning; last dose on Day 12 in the morning.

Group II

Day 1: One dose of 3.6 mg delta-9-THC or placebo administered in the morning.

Days 5-19: Multiple dose administration (3.6 mg delta-9-THC three times daily—every 8 hours); first dose on Day 5 in the morning; last dose on Day 19 in the morning. Compositions are shown in Table 1 above.

For both dose levels, the first and the last dose of study drug was given under fasted conditions. Subjects were confined to the study site from the evening of Day −2 (Day 1 is the day of administration of the first dose of study drug) until the 120-hour blood sample following the final dose of study drug, resulting in an 18-day confinement period for subjects in Group I and a 25-day confinement period for Group II. Between the completion of Group I and the start of Group II, an interim safety and pharmacokinetic analysis was performed. Based on the results of the interim analyses, the dose level for investigation in Group II was determined.

Two MDI dosage strengths were used corresponding to the same formulations used in Example 1: one delivering 0.3 mg delta-9-THC (or placebo) per actuation and one delivering 1.2 mg delta-9-THC (or placebo) per actuation. The MDI consisted of a pressurized (via propellants) container and a metered-dose valve. The propellants provided the necessary force to expel the drug, and also acted as a solvent and diluent. The canister unit was provided within a mouthpiece (oral adapter), to expel an exact amount of drug, in the proper particle size distribution, upon each actuation. Two basic formulations containing 0.3 and 1.2 mg of delta-9-THC per 50 μL were developed with propellant 1,1,1,2 tetrafluoroethane 134a (HFA 134a), and ethanol as solvent. The formulations were made in accordance with the composition used in Example 1.

Safety was measured by monitoring adverse events, physical examination, clinical laboratory and pulmonary function tests, vital signs, 12 lead ECG, and telemetry. Various pharmacokinetic and pharmacodynamic measurements and sampling were taken according to the assessment schedule in Tables 2-3:

TABLE 2
Assessment Schedule
GROUP I

| | Day(s) | | | | | | | | | | | | | | | | | | | | | | | |
|---|---|---|---|---|---|---|---|---|---|---|---|---|---|---|---|---|---|---|---|---|---|---|---|---|
| | S⁵ | −2 | −1 | 1 and 12 | | | | | | | | | | | | | | | | | 2-4 | 5-11 | 13-17 | FU⁶ |
| | | | | Minutes/Hours | | | | | | | | | | | | | | | | | | | | |
| | | | | P⁴ | 0 | 2 | 5 | 10 | 15 | 20 | 30 | 40 | 1 | 2 | 3 | 4 | 6 | 8 | 10 | 16 | | | | |
| Screening | X | | | | | | | | | | | | | | | | | | | | | | | |
| Informed Consent | X | | | | | | | | | | | | | | | | | | | | | | | |
| Inc./Exclusion | X | | X | | | | | | | | | | | | | | | | | | | | | |
| Criteria | | | | | | | | | | | | | | | | | | | | | | | | |
| Medical/Drug | X | | | | | | | | | | | | | | | | | | | | | | | |
| History | | | | | | | | | | | | | | | | | | | | | | | | |
| Physical | X | | | | | | | | | | | | | | | | | | | | X⁸ | | X⁸ | X |
| Examination | | | | | | | | | | | | | | | | | | | | | | | | |
| CBC, Clinical | X | | X | | | | | | | | | | | | | | | | | | X⁸ | | X⁸ | X |
| Chemistry | | | | | | | | | | | | | | | | | | | | | | | | |
| Viral Screen | X | | | | | | | | | | | | | | | | | | | | | | | |
| Urinalysis | X | | X | | | | | | | | | | | | | | | | | | X⁸ | | X⁸ | X |
| Urine Pregnancy | | | X | | | | | | | | | | | | | | | | | | | | | X |
| Test (females | | | | | | | | | | | | | | | | | | | | | | | | |
| only) | | | | | | | | | | | | | | | | | | | | | | | | |
| Drugs of abuse | X | | X | | | | | | | | | | | | | | | | | | | | | |
| (urinary) | | | | | | | | | | | | | | | | | | | | | | | | |
| Alcohol blood | X | | X | | | | | | | | | | | | | | | | | | | | | |
| test | | | | | | | | | | | | | | | | | | | | | | | | |
| Admit to Clinic | | X | | | | | | | | | | | | | | | | | | | | | | |
| ECG Recording | X | | X¹⁴ | X | | | | X | | X | | X | X | X | | X | X | | X | | X⁸ | | | X |
| Heart rate by | | | | | | X | X | | X | | X | | | | | | | | | | | | | |
| telemetrics¹ | | | | | | | | | | | | | | | | | | | | | | | | |
| Heart Rate by | X | | | X | | | | X | | X | | X | X | X | X | X | X | X | X | | X⁹ | X¹² | X⁹ | X |
| blood pressure | | | | | | | | | | | | | | | | | | | | | | | | |
| Blood Press./ | X | | | X | | | | X | | X | | X | X | X | X | X | X | X | X | | X⁹ | X¹² | X⁹ | X |
| Res. Rate⁷ | | | | | | | | | | | | | | | | | | | | | | | | |
| Oral temp | X | | | X | | | | | | X | | | X | | X | | | | | | X⁹ | X¹² | X⁹ | X |
| Pulmonary | X | | | X | | | | | | | | | X | | | X | | | | | X⁸ | X¹² | X⁸ | X |
| Function | | | | | | | | | | | | | | | | | | | | | | | | |
| Cognition Test | | | X | | | | | | | | | | | | | | | | | | | | | |
| Training | | | | | | | | | | | | | | | | | | | | | | | | |
| Perform | | | | X | | | | | | X² | | | X | | | | | | X | | X⁸ | | X⁸ | |
| Cognition Tests | | | | | | | | | | | | | | | | | | | | | | | | |
| Conjunctiva | | | | | | | | X | | X | X | | X | | | X | | | X | | X⁸ | | X⁸ | |
| Congestion | | | | | | | | | | | | | | | | | | | | | | | | |
| Visual Analogue | | | | X | | | X | | X | | X | | X | X | | X | | | X | | X⁸ | | X⁸ | |
| Scales + SDR³ | | | | | | | | | | | | | | | | | | | | | | | | |
| Addiction Res. | | | | X | | | | | | | | | X | | | | | | X | | X⁸ | | X⁸ | |
| Inventory | | | | | | | | | | | | | | | | | | | | | | | | |
| MDI training | | | X | | | | | | | | | | | | | | | | | | | | | |
| DOSING | | | | | X¹⁰ | | | | | | | | | | | | | | | | | X¹⁰ | | |
| Adverse Event | | | | | | | | | | | | | | | | | | | | | | | | |
| Recording | | | | | | | | | | | | | | | | | | | | | | | | |
| Concomitant | | | | | | | | | | | | | | | | | | | | | | | | |
| Medication | | | | | | | | | | | | | | | | | | | | | | | | |
| Blood | | | | | X | | X | X | X | X | X | X | X | X | X | X | X | X | X | X | X | X¹¹ | X | |
| Pharmacokinetic | | | | | | | | | | | | | | | | | | | | | | | | |
| Discharge from | | | | | | | | | | | | | | | | | | | | | | | X¹³ | |
| Clinic | | | | | | | | | | | | | | | | | | | | | | | | |

¹Continuous telemetry monitoring 1 h pre-dose to 2 h post-dose on Days 1 and 12.
²Adapted test of two minutes at 20 minutes post-dose.
³Visual Analogue Scales together with Subjective Drug Rating.
⁴Pre-dose.
⁵Screening period (days −21 to 1).
⁶Follow up visit (7 to 14 days after dosing in the last period).
⁷From dosing to 2 hours (inclusive) post-dose blood pressures was recorded in semi-recumbent position. Pre-dose and after 2 hours post-dose blood pressures was recorded in supine position.
⁸At 24 hours post-dose only.
⁹At 24 hours post-dose and then once daily in the morning on the other Days.
¹⁰On Days 1 and 12 dosing was only in the morning. On other dosing days drug was administered three times daily (every 8 hours).
¹¹Pre-dose samples was obtained on Days 7-11 prior to morning dose administration.
¹²Obtained once daily in the morning.
¹³At 120 hours post-dose subjects was discharged.
¹⁴A total of 6 ECGs was obtained from each subject on Day-1 at approximately 10:00, 15:30, and 20:30. Two ECGs (5 minutes apart) was taken at each time.

TABLE 3
Assessment Schedule
GROUP II

| | Day(s) | | | | | | | | | | | | | | | | | | | | | | | |
|---|---|---|---|---|---|---|---|---|---|---|---|---|---|---|---|---|---|---|---|---|---|---|---|---|
| | S⁵ | −2 | −1 | 1 and 19 | | | | | | | | | | | | | | | | | 2-4 | 5-18 | 20-24 | FU⁶ |
| | | | | Minutes/Hours | | | | | | | | | | | | | | | | | | | | |
| | | | | P⁴ | 0 | 2 | 5 | 10 | 15 | 20 | 30 | 40 | 1 | 2 | 3 | 4 | 6 | 8 | 10 | 16 | | | | |
| Screening | X | | | | | | | | | | | | | | | | | | | | | | | |
| Informed Consent | X | | | | | | | | | | | | | | | | | | | | | | | |
| Inc./Exclusion | X | | X | | | | | | | | | | | | | | | | | | | | | |
| Criteria | | | | | | | | | | | | | | | | | | | | | | | | |
| Medical/Drug | X | | | | | | | | | | | | | | | | | | | | | | | |
| History | | | | | | | | | | | | | | | | | | | | | | | | |
| Physical | X | | | | | | | | | | | | | | | | | | | | X⁸ | | X⁸ | X |
| Examination | | | | | | | | | | | | | | | | | | | | | | | | |
| CBC, Clinical | X | | X | | | | | | | | | | | | | | | | | | X⁸ | | X⁸ | X |
| Chemistry | | | | | | | | | | | | | | | | | | | | | | | | |
| Viral Screen | X | | | | | | | | | | | | | | | | | | | | | | | |
| Urinalysis | X | | X | | | | | | | | | | | | | | | | | | X⁸ | | X⁸ | X |
| Urine Pregnancy | | | X | | | | | | | | | | | | | | | | | | | | | X |
| Test (females | | | | | | | | | | | | | | | | | | | | | | | | |
| only) | | | | | | | | | | | | | | | | | | | | | | | | |
| Drugs of abuse | X | | X | | | | | | | | | | | | | | | | | | | | | |
| (urinary) | | | | | | | | | | | | | | | | | | | | | | | | |
| Alcohol blood | X | | X | | | | | | | | | | | | | | | | | | | | | |
| test | | | | | | | | | | | | | | | | | | | | | | | | |
| Admit to Clinic | | X | | | | | | | | | | | | | | | | | | | | | | |
| ECG Recording | X | | X¹⁴ | X | | | | X | | X | | X | X | X | | X | X | | X | | X⁸ | | | X |
| Heart rate by | | | | | | X | X | | X | | X | | | | | | | | | | | | | |
| telemetrics¹ | | | | | | | | | | | | | | | | | | | | | | | | |
| Heat Rate by | X | | | X | | | | X | | X | | X | X | X | X | X | X | X | X | | X⁹ | X¹² | X⁹ | X |
| blood pressure | | | | | | | | | | | | | | | | | | | | | | | | |
| Blood Press./ | X | | | X | | | | X | | X | | X | X | X | X | X | X | X | X | | X⁹ | X¹² | X⁹ | X |
| Res. Rate⁷ | | | | | | | | | | | | | | | | | | | | | | | | |
| Oral temp | X | | | X | | | | | | X | | | X | | X | | | | | | X⁹ | X¹² | X⁹ | X |
| Pulmonary | X | | | X | | | | | | | | | X | | | X | | | | | X⁸ | X¹² | X⁸ | X |
| Function | | | | | | | | | | | | | | | | | | | | | | | | |
| Cognition Test | | | X | | | | | | | | | | | | | | | | | | | X¹⁵ | | |
| Training | | | | | | | | | | | | | | | | | | | | | | | | |
| Perform | | | | X | | | | | | X² | | | X | | | | | | X | | X⁸ | | X⁸ | |
| Cognition Tests | | | | | | | | | | | | | | | | | | | | | | | | |
| Conjunctiva | | | | | | | | X | | X | X | | X | | | X | | | X | | X⁸ | | X⁸ | |
| Congestion | | | | | | | | | | | | | | | | | | | | | | | | |
| Visual Analogue | | | | X | | | X | | X | | X | | X | X | | X | | | X | | X⁸ | | X⁸ | |
| Scales + SDR³ | | | | | | | | | | | | | | | | | | | | | | | | |
| Addiction Res. | | | | X | | | | | | | | | X | | | | | | X | | X⁸ | | X⁸ | |
| Inventory | | | | | | | | | | | | | | | | | | | | | | | | |
| MDI training | | | X | | | | | | | | | | | | | | | | | | | | | |
| DOSING | | | | | X¹⁰ | | | | | | | | | | | | | | | | | X¹⁰ | | |
| Adverse Event | | | | | | | | | | | | | | | | | | | | | | | | |
| Recording | | | | | | | | | | | | | | | | | | | | | | | | |
| Concomitant | | | | | | | | | | | | | | | | | | | | | | | | |
| Medication | | | | | | | | | | | | | | | | | | | | | | | | |
| Blood | | | | X | | X | X | X | X | X | X | X | X | X | X | X | X | X | X | X | X | X¹¹ | X | |
| Pharmacokinetic | | | | | | | | | | | | | | | | | | | | | | | | |
| Discharge from | | | | | | | | | | | | | | | | | | | | | | | X | |
| Clinic | | | | | | | | | | | | | | | | | | | | | | | | |

¹Continuous telemetry monitoring 1 h pre-dose to 2 h post-dose on Days 1 and 19.
²Adapted test of two minutes at 20 minutes post-dose.
³Visual Analogue Scales together with Subjective Drug Rating.
⁴Pre-dose.
⁵Screening period (days −21 to 1).
⁶Follow up visit (7 to 14 days after dosing in the last period).
⁷From dosing to 2 hours (inclusive) post-dose blood pressures was recorded in semi-recumbent position. Pre-dose and after 2 hours post-dose blood pressures was recorded in supine position.
⁸At 24 hours post-dose only.
⁹At 24 hours post-dose and then once daily in the morning on the other Days.
¹⁰On Days 1 and 19 dosing was only in the morning. On other dosing days drug was administered three times daily (every 8 hours).
¹¹Pre-dose samples was obtained on Days 7-18 prior to morning dose administration.
¹²Obtained once daily in the morning.
¹³At 120 hours post-dose subjects was discharged.
¹⁴A total of 6 ECGs was obtained from each subject on Day-1 at approximately 10:00, 15:30, and 20:30. Two ECGs (5 minutes apart) was taken at each time.
¹⁵Subjects were re-trained on the CDR system on Day 18.

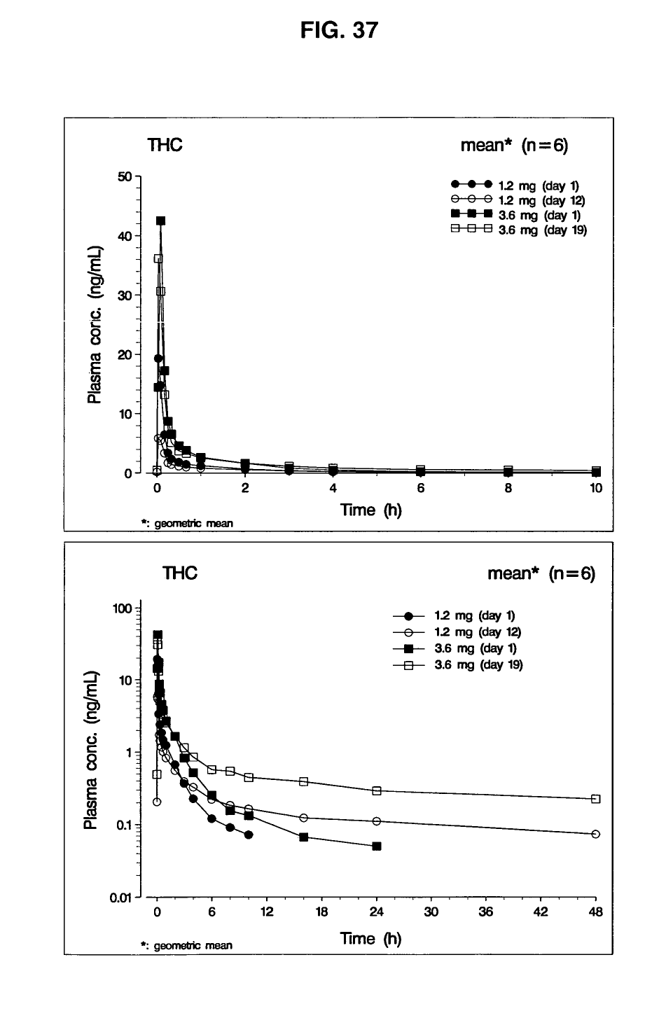

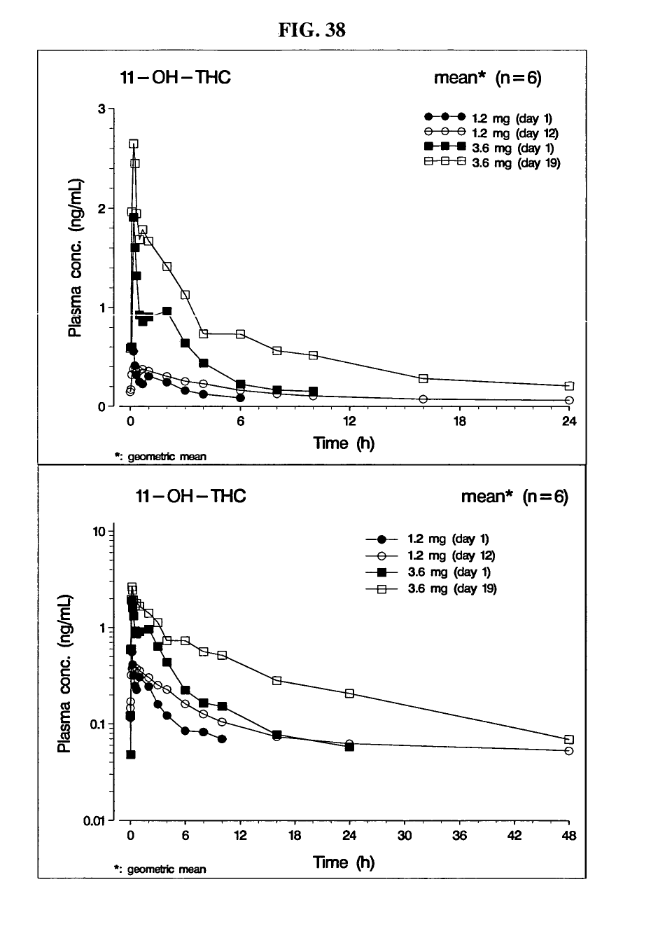

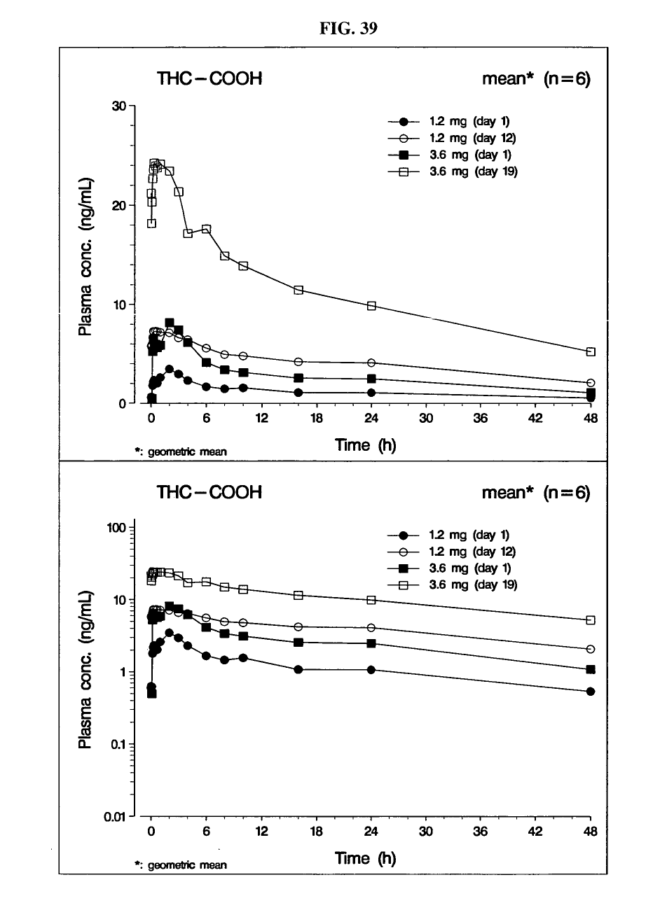

Linear and semi-logarithmic geometric mean plasma concentration versus time profiles of delta-9-THC, 11-OH-THC and THC-COOH per treatment are presented in FIG. 37, FIG. 38, and FIG. 39, respectively. A summary of the pharmacokinetic data for Groups I-II are provided in Tables 5-6.

TABLE 5
Summary of pharmacokinetic parameters for delta-9-THC, 11-OH-THC
and THC-COOH after dosing with 1.2 mg delta-9-THC

| | | | |
|---|---|---|---|
| | | Geometric | Arithmetic |
| Treatment | Parameter | Mean (Range)* | Mean (SD) |

Delta-9-THC

| | | | |
|---|---|---|---|
| 1.2 mg s.d. | Cₘₐₓ (ng/mL) | 19.7 (4.61-35.0) | 24.0 (12.3) |
| | tₘₐₓ (h) | 0.03 (0.03-0.08) | 0.04 (0.02) |
| | AUC₍₀₋₈₎ (ng · h/mL) | 6.10 (3.02-9.08) | 6.61 (2.61) |
| | AUC_{(0-inf)} (ng · h/mL) | 6.55 (3.04-9.59) | 7.14 (2.85) |
| | t_{½}(h) | 3.39 (1.12-7.89) | 4.02 (2.35) |
| 1.2 mg m.d. | Cₘₐₓ (ng/mL) | 6.90 (0.31-24.8) | 12.3 (9.35) |
| | tₘₐₓ (h) | 0.06 (0.03-0.08) | 0.06 (0.03) |
| | AUC₍₀₋₈₎ (ng · h/mL) | 4.52 (1.03-12.2) | 5.74 (3.79) |
| | t_{½}(h) | 25.3 (12.4-57.8) | 29.1 (16.8) |

11-OH-THC

| | | | |
|---|---|---|---|
| 1.2 mg s.d. | Cₘₐₓ (ng/mL) | 0.63 (0.10-1.55) | 0.86 (0.57) |
| | tₘₐₓ (h) | 0.17 (0.17-2.00) | 0.48 (0.75) |
| | AUC₍₀₋₈₎ (ng · h/mL) | 1.26 (0.41-2.66) | 1.50 (0.88) |
| | AUC_{(0-inf)} (ng · h/mL) | 1.56 (0.49-3.39) | 1.93 (1.22) |
| | t_{½}(h) | 3.91 (2.55-6.01) | 4.19 (1.67) |
| 1.2 mg m.d. | Cₘₐₓ (ng/mL) | 0.54 (0.26-1.26) | 0.61 (0.36) |
| | tₘₐₓ (h) | 0.42 (0.17-1.00) | 0.48 (0.36) |
| | AUC₍₀₋₈₎ (ng · h/mL) | 1.89 (1.13-3.48) | 2.07 (0.99) |
| | t_{½}(h) | 13.9 (7.41-25.2) | 14.9 (6.08) |

THC-COOH

| | | | |
|---|---|---|---|
| 1.2 mg s.d. | Cₘₐₓ (ng/mL) | 3.77 (2.81-4.40) | 3.81 (0.56) |
| | tₘₐₓ (h) | 2.00 (0.25-2.00) | 1.54 (0.75) |
| | AUC₍₀₋₈₎ (ng · h/mL) | 18.2 (13.0-24.2) | 18.5 (3.85) |
| | AUC_{(0-inf)} (ng · h/mL) | 82.0 (60.5-110) | 83.6 (18.5) |
| | t_{½}(h) | 32.0 (23.9-50.6) | 33.9 (12.8) |
| 1.2 mg m.d. | Cₘₐₓ (ng/mL) | 7.97 (6.03-10.9) | 8.15 (1.87) |
| | tₘₐₓ (h) | 0.33 (0.25-2.00) | 0.86 (0.88) |
| | AUC₍₀₋₈₎ (ng · h/mL) | 50.1 (34.1-69.8) | 51.9 (14.7) |
| | t_{½}(h) | 31.0 (23.2-43.8) | 32.1 (9.07) |

s.d. = single dose profile measured on Day 1
m.d. = multiple dose profile measured on Day 12 in Group I
*for tₘₐₓ the median and the range are presented

TABLE 6
Summary of pharmacokinetic parameters for delta-9-THC, 11-
OH-THC and THC-COOH after dosing with 3.6 mg delta-9-THC

| | | | |
|---|---|---|---|
| | | Geometric | |
| Treatment | Parameter | Mean (Range)* | Arithmetic Mean (SD) |

Delta-9-THC

| | | | |
|---|---|---|---|
| 3.6 mg s.d. | Cₘₐₓ (ng/mL) | 42.5 (27.1-63.2) | 44.7 (15.1) |
| | tₘₐₓ (h) | 0.08 (0.08-0.08) | 0.08 (0.00) |
| | AUC₍₀₋₈₎ | 14.0 (9.02-19.2) | 14.7 (4.63) |
| | (ng · h/mL) | | |
| | AUC_{(0-inf)} | 15.6 (9.82-21.1) | 16.3 (4.84) |
| | (ng · h/mL) | | |
| | t_{½}(h) | 6.01 (3.32-10.8) | 6.41 (2.52) |
| 3.6 mg m.d. | Cₘₐₓ (ng/mL) | 37.4 (16.9-58.4) | 40.4 (15.4) |
| | tₘₐₓ (h) | 0.03 (0.03-0.08) | 0.04 (0.02) |
| | AUC₍₀₋₈₎ | 14.9 (9.03-22.3) | 15.8 (5.47) |
| | (ng · h/mL) | | |
| | t_{½}(h) | 92.9 (59.6-110) | 94.8 (18.9) |

11-OH-THC

| | | | |
|---|---|---|---|
| 3.6 mg s.d. | Cₘₐₓ (ng/mL) | 1.91 (1.12-4.38) | 2.21 (1.37) |
| | tₘₐₓ (h) | 0.17 (0.17-0.20) | 0.18 (0.01) |
| | AUC₍₀₋₈₎ | 4.44 (2.22-9.46) | 5.15 (3.04) |
| | (ng · h/mL) | | |
| | AUC_{(0-inf)} | 5.86 (3.03-11.6) | 6.82 (3.98) |
| | (ng · h/mL) | | |
| | t_{½}(h) | 5.44 (2.96-11.0) | 5.98 (2.91) |
| 3.6 mg m.d. | Cₘₐₓ (ng/mL) | 2.87 (1.27-6.97) | 3.40 (2.17) |
| | tₘₐₓ (h) | 0.17 (0.17-0.67) | 0.27 (0.20) |
| | AUC₍₀₋₈₎ | 8.69 (4.06-19.8) | 10.1 (6.13) |
| | (ng · h/mL) | | |
| | t_{½}(h) | 39.5 (5.65-94.2) | 57.3 (37.9) |

THC-COOH

| | | | |
|---|---|---|---|
| 3.6 mg s.d. | Cₘₐₓ (ng/mL) | 8.34 (6.01-14.4) | 8.75 (3.16) |
| | tₘₐₓ (h) | 2.00 (2.00-3.00) | 2.33 (0.52) |
| | AUC₍₀₋₈₎ | 44.5 (30.2-74.4) | 47.1 (17.5) |
| | (ng · h/mL) | | |
| | AUC_{(0-inf)} | 152 (64.8-283) | 172 (85.6) |
| | (ng · h/mL) | | |
| | t_{½}(h) | 21.7 (11.3-39.2) | 23.8 (11.3) |
| 3.6 mg m.d. | Cₘₐₓ (ng/mL) | 25.5 (13.2-45.0) | 28.1 (12.7) |
| | tₘₐₓ (h) | 0.75 (0.33-2.00) | 1.03 (0.79) |
| | AUC₍₀₋₈₎ | 159 (75.5-317) | 182 (97.5) |
| | (ng · h/mL) | | |
| | t_{½}(h) | 37.4 (24.0-62.3) | 39.6 (15.3) |

s.d. = single dose profile measured on Day 1
m.d. = multiple dose profile measured on Day 19 in Group II
*for tₘₐₓ the median and the range are presented

The plasma concentration-time curves of delta-9-THC demonstrated at least a bi-phasic elimination profile, with the initial elimination phase being slower after multiple compared to single dosing for both dose levels. On Day 1 of both dose levels, the terminal elimination half-life could not be determined accurately, because of the limited number of samples over time showing concentrations above the limit of quantification (“LOQ”) in the majority of subjects. As a result, AUC_{(0-inf)} values (Table 5) were underestimated.

The plasma concentration-time curves of 11-OH-THC also demonstrated at least a bi-phasic elimination profile with slower initial elimination after multiple compared to single dosing. Again, the limited number of samples over time with concentrations above LOQ hindered accurate estimation of terminal elimination half-lives except after multiple dosing at the 3.6 mg dose level. In addition, the individual plasma concentration-time curves were characterized by a second peak, between 10 minutes and 4 hours after dosing.

The plasma concentration-time curves of THC-COOH showed almost no distribution phase and a multi-phasic elimination phase, especially after multiple dosing. Concentrations were above LOQ for sufficient periods of time to allow adequate calculation of terminal elimination half-lives, after both single and multiple dose administration.

For both dose levels, rapid systemic absorption of delta-9-THC was observed, with Tₘₐₓ ranging between 0.03 and 0.08 hours (2-5 minutes) after both single and multiple dose administration. Plasma concentrations for delta-9-THC metabolites, 11-OH-THC and THC-COOH, peaked later than the parent compound, i.e., between 10 minutes and 2 hours post-dose for 11-OH-THC and between 15 minutes and 3 hours post-dose for THC-COOH. Tₘₐₓ values were variable between subjects for 11-OH-THC and THC-COOH as demonstrated by the wide ranges.

Dose-related increases in Cₘₐₓ and AUC values were observed for delta-9-THC, 11-OH-THC, and THC-COOH after both single and multiple dose administration at both dose levels.

Maximum concentrations of 11-OH-THC were approximately 25-fold lower compared to the parent compound after single dosing, and 13-fold lower after multiple dosing. THC-COOH Cₘₐₓ values were similar to the parent after 1.2 mg multiple dosing. In contrast, THC-COOH Cₘₐₓ values were 5-fold lower than delta-9-THC after 1.2 mg and 3.6 mg single dose administration.

Based on AUC_{(0-inf)} after single dosing and AUC₍₀₋₈₎ after multiple dosing, the following trends in exposure were observed. After administration of delta-9-THC, exposure to the 11-OH-THC active metabolite was approximately four-fold lower compared to the parent drug following single dose administration, and approximately two-fold lower after multiple dosing. After both single and multiple dose delta-9-THC administration, exposure to the THC-COOH metabolite was approximately 10-fold higher compared to the parent. These trends were evident at both dose levels studied.

Due to a limited number of detectable plasma concentrations after single dose administration the terminal elimination half-life values are best estimated after multiple dosing. Increases in terminal elimination half-lives of delta-9-THC and 11-OH-THC were observed with multiple dosing and with higher dose exposure.

Statistical Analysis of Pharmacokinetics.

In Table 7, the geometric mean ratios and 90% confidence intervals used to evaluated dose proportionality are provided. The pharmacokinetic parameters were not found to deviate significantly after single or multiple dose administration for delta-9-THC, 11-OH-THC, or THC-COOH.

TABLE 7
Results of explorative testing on dose proportionality of PK parameters

| Treatment | Parameter | Delta-9-THC | 11-OH-THC | THC-COOH |
|---|---|---|---|---|
| Single dose | AUC₍₀₋₈₎ | 0.77 (0.50-1.18)* | 1.18 (0.60-2.33) | 0.82 (0.60-1.11) |
| | AUC_{(0-inf)} | 0.79 (0.51-1.23) | 1.25 (0.61-2.57) | 0.62 (0.39-0.98) |
| | AUC₍₀₋ₜ₎ | 0.81 (0.52-1.25) | 1.43 (0.64-3.20) | 0.61 (0.34-1.09) |
| | Cₘₐₓ | 0.71 (0.38-1.37) | 1.01 (0.42-2.42) | 0.74 (0.56-0.96) |
| Multiple dose | AUC₍₀₋₈₎ | 1.10 (0.55-2.18) | 1.53 (0.87-2.69) | 1.05 (0.65-1.71) |
| | Cₘₐₓ | 1.81 (0.52-6.24) | 1.79 (0.95-3.36) | 1.07 (0.71-1.60) |

*Point estimates of geometric mean ratios (3.6 mg:1.2 mg dose levels) of dose-normalized pharmacokinetic parameters, after backtransformation from contrasts on log scale; 90% confidence intervals in parentheses.

Accumulation ratios, calculated as AUC₍₀₋₈₎ after multiple dosing compared to AUC₍₀₋₈₎ after single dose administration, and ratios of AUC_{(0-inf)} after single dosing compared to AUC₍₀₋₈₎ after multiple dose administration are presented in Table 8 below.

For delta-9-THC, no statistically significant accumulation was observed during multiple dosing. This is in apparent contrast with the long terminal elimination half-life (24 hours at 1.2 mg, 93 hours at 3.6 mg) and the 8-hours dosing interval, indicating the long half-life is not judged to be clinically relevant. Ratios of greater than one for AUC₍₀₋₈₎ were observed with 11-OH-THC and THC-COOH for multiple dosing compared to single dose administration, indicating that accumulation occurred during multiple dosing.

TABLE 8
Summary statistics of the ratios multiple dose/single dose for
AUCs of delta-9-THC, 11-OH-THC and THC-COOH

| | |
|---|---|
| Ratio | |
| AUC₍₀₋₈₎ m.d./ | Ratio |
| AUC₍₀₋₈₎ s.d.* | AUC₍₀₋₈₎ m.d./ |
| (Accumulation | AUC_{(0-inf)} s.d. |
| factor) | (Linearity) |

| Analyte | Mean | Min-Max | Mean | Min-Max |
|---|---|---|---|---|
| Group I, 1.2 mg | | | | |
| Delta-9-THC | 0.85 | 0.26-1.41 | 0.79 | 0.25-1.32 |
| 11-OH-THC | 1.59 | 0.98-2.75 | 1.30 | 0.73-2.30 |
| THC-COOH | 2.90 | 1.72-4.23 | 0.65 | 0.36-0.91 |
| Group II, 3.6 mg | | | | |
| Delta-9-THC | 1.15 | 0.47-1.91 | 1.02 | 0.43-1.55 |
| 11-OH-THC | 2.10 | 1.05-2.97 | 1.55 | 0.87-2.09 |
| THC-COOH | 3.92 | 1.77-5.68 | 1.11 | 0.56-1.60 |

*s.d.: single dose;
m.d.: multiple dose

The ratios of AUC₍₀₋₈₎ after multiple dosing versus AUC_{(0-inf)} after single dose administration did not deviate clearly or significantly from unity in case of delta-9-THC and THC-COOH, suggesting the multiple dose pharmacokinetics are linear.

Achievement of steady state was analyzed using explorative statistical analysis. Steady state concentrations of delta-9-THC and 11-OH-THC were apparently reached after six days of dosing at 1.2 mg delta-9-THC, but trough concentrations for THC-COOH showed significant differences between days over the last week of dosing, suggesting lack of steady state for this metabolite. With respect to the 3.6-mg dose level, statistical analysis suggested that steady state was achieved after seven or eight days of dosing in case of the active metabolite, 11-OH-THC, and the inactive metabolite, THC-COOH, but not for delta-9-THC. Visual inspection of the delta-9-THC profile showed that trough concentrations of delta-9-THC increased more or less continuously from Day 7 until Day 17, followed by a sharp decrease on Days 18 and 19.

Pharmacodynamic Analysis

Heart Rate

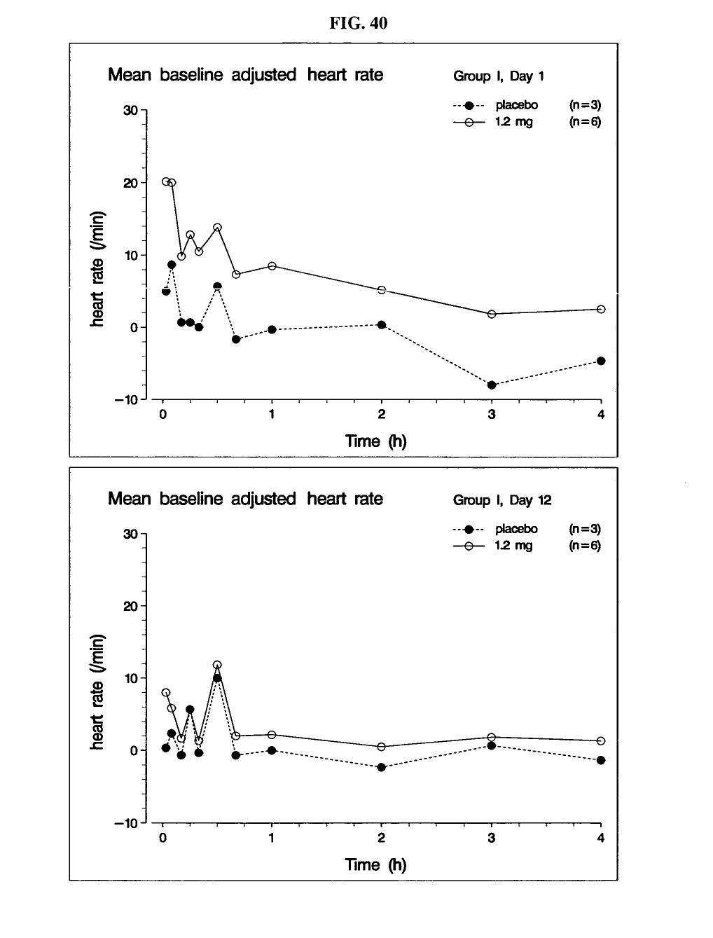

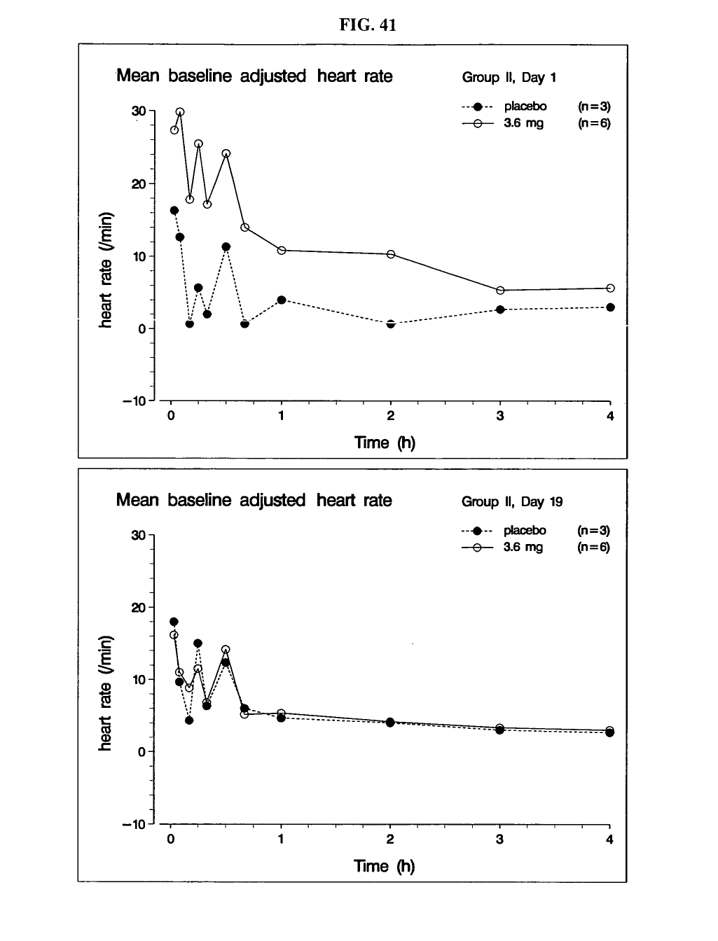

As shown in FIGS. 40-41, mean baseline-adjusted heart rate increased immediately, and tended to remain higher during the first 2 hours after the first dose of delta-9-THC on Day 1 compared to placebo, at both dose levels. This effect was greater and slightly longer in duration at the 3.6 mg dose level compared to the 1.2 mg dose level. Mean increase above baseline showed values between 10 and 20 bpm during the first 0.5 hour after dosing with 1.2 mg delta-9-THC, compared to a mean increase above baseline of less than 10 bpm in placebo subjects. After 3.6 mg delta-9-THC, mean increase of heart rate above baseline was between 17 and 30 bpm in the first 0.5 hour after dosing, compared to mean values between one and 16 bpm in placebo subjects. Between 0.5 and 2 hours after administration of 3.6 mg delta-9-THC, mean increase in heart rate above baseline showed values between 10 and 14 bpm, compared to less than five bpm in subjects receiving placebo.

There were no obvious differences in baseline-adjusted heart rate between active drug and placebo treatment for the remainder of the study in either group, which may be expected since these assessments were performed more than 6 hours after the previous dose. On the last day of dosing, when heart rate was measured before and on several occasions after dosing, baseline-adjusted heart rate in subjects receiving delta-9-THC increased to a lesser extent compared to the first day of dosing. Mean increase above baseline was ≦12 bpm in Group I (at 0.5 hours after dosing) and ≦18 bpm in Group II (at 5 minutes after dosing). The increase in heart rate following delta-9-THC treatment leveled off in the course of multiple dosing. Separation from placebo was only evident to a very limited extent after multiple dosing with 1.2 mg delta-9-THC, and no longer evident with 3.6 mg (FIG. 5).

It should be mentioned that mean increases of baseline-adjusted heart rate as obtained from telemetric monitoring (2, 5, 15 and 30 minutes after dosing) were always higher than those taken from vitals signs measurements in between and after the telemetric readings (10, 20 and 40 minutes after dosing), in all treatment groups and on both the first and the last days of dosing.

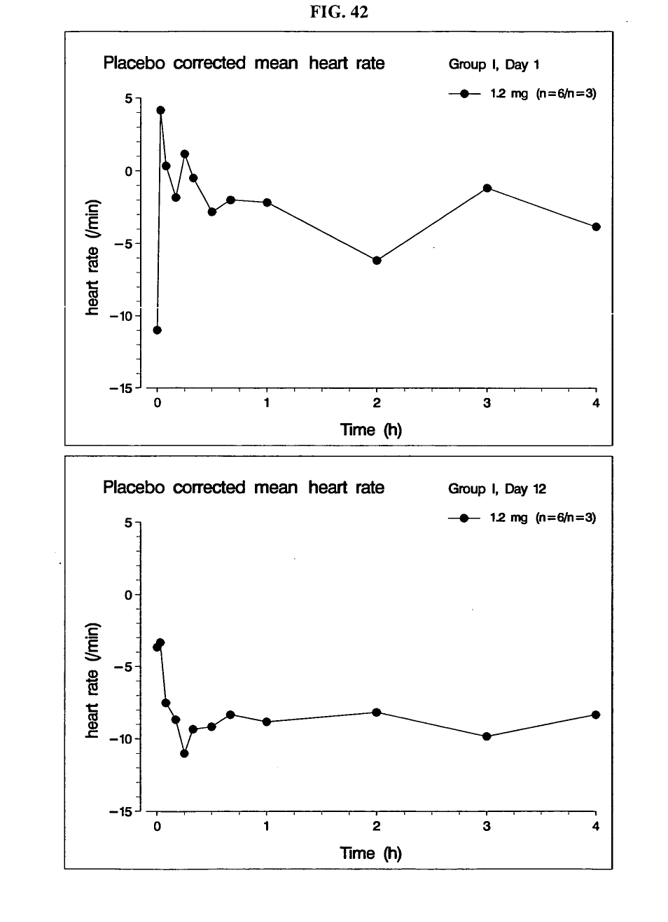

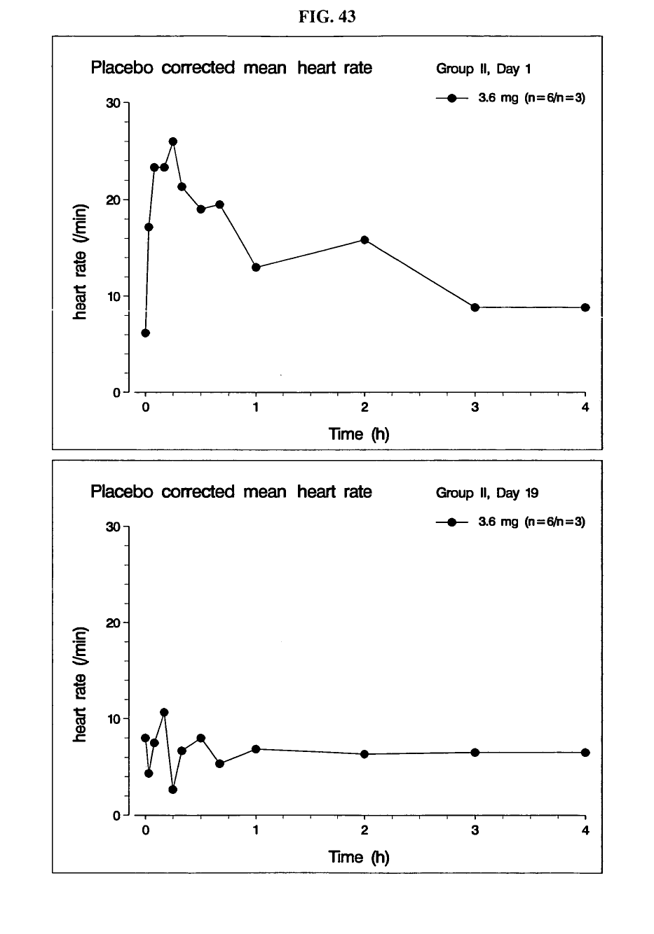

When expressed as placebo-corrected values (FIGS. 42-43), there was a limited and short-lasting increase in mean heart rate after 1.2 mg delta-9-THC on Day 1, and no increase on Day 12. However, mean placebo-corrected heart rate was clearly elevated after 3.6 mg delta-9-THC, with maximum values reaching between 20 and 30 bpm increase by 5-20 minutes post-dose on Day 1 and the increase lasting until at least 4 hours post-dose. Similar to baseline-adjusted heart rate, the increase was much smaller after 19 days of dosing with 3.6 mg delta-9-THC (maximum increase approximately 10 bpm by 10 minutes post-dose), but apparent duration was similar to Day 1.

Conjunctiva Congestion

In Groups I and II combined, only one subject (1.2 mg delta-9-THC), showed slight conjunctiva congestion on Day 12, i.e., the last day of dosing. This was observed at 10 minutes after dosing on Day 12, and resolved within 4 hours post-dose. No other observation of conjunctiva congestion was reported throughout the study.

Bond-Lader VAS

Alertness

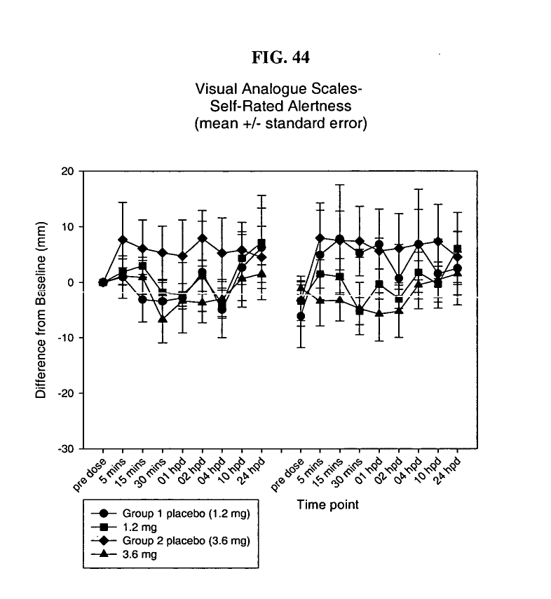

The data for Self-rated Alertness (FIG. 44) showed some indications of change in performance over the study, with some improvements in performance for placebo over Day 1. Following multiple dosing, alertness declined for placebo at pre-dose on the final day of dosing, but then subsequently recovered. Less change was seen for the active doses, though there was some indication of slight transient declines on each day. The primary analysis showed a significant dose-day interaction (p≦0.01). The comparisons showed no decrements for both 1.2 mg (p>0.05) and 3.6 mg (p>0.05) against placebo, on the last day of dosing. The secondary analysis showed no significant effect of dose or dose-day interaction.

Contentment

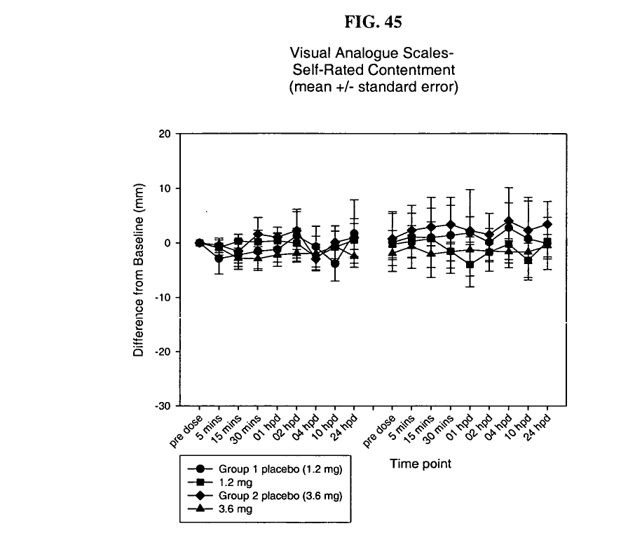

The data for Self-rated Contentment (FIG. 45) showed little indication of change in performance over the study. The primary analysis showed a significant dose*day interaction (p≦0.01). The comparisons showed no support for differences between the doses on either day. The interaction resulted from slight changes in the magnitude of shift for placebo and 3.6 mg, while 1.2 mg improved slightly on Day 1 and declined slightly on Day 12. The secondary analysis showed no significant effect of dose or dose*day interaction.

Calmness

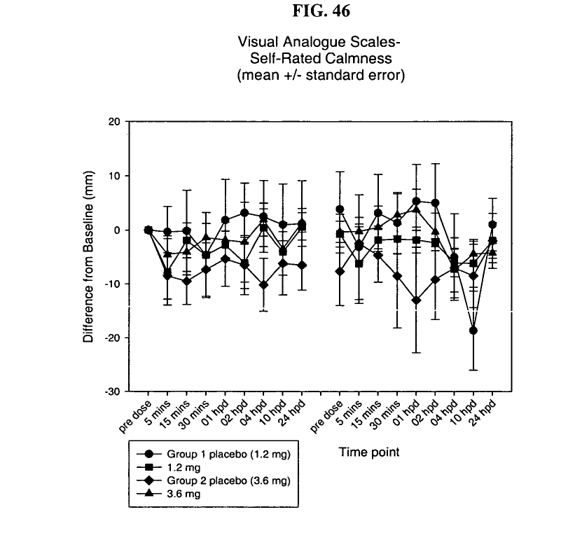

The data for Self-rated Calmness (FIG. 46) showed some fluctuation in performance over the study, but no clear pattern emerged. The primary analysis showed no significant effect of dose, or interaction between dose and day and/or time. The secondary analysis showed no significant effect of dose or dose*day interaction.

Cognitive Test Battery

Simple Reaction Time

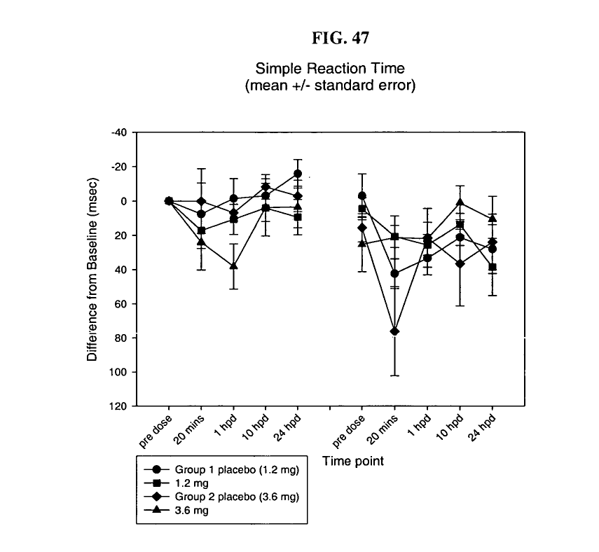

The data for Simple Reaction Time (FIG. 47) showed some fluctuation in performance over the study, with no indication of separation between the active doses and placebo. However, some slight indication of a decline at 1 hour post-dose was seen on Day 1 for 3.6 mg, whilst there was some indication of a 20 minutes post-dose decline for placebo on the final Day of dosing (Day 12/19).

The primary analysis showed a significant dose*day interaction (p<0.01). The comparisons did not show any significant differences between the different doses on either day. The interaction was most clearly the result of a shift from poorer performance with 3.6 mg than placebo on Day 1 and better performance than placebo on Day 19, in part due to a final Day (Day 12/19) decline with placebo. The secondary analysis showed no significant effect of dose or dose*day interaction.

Choice Reaction Time

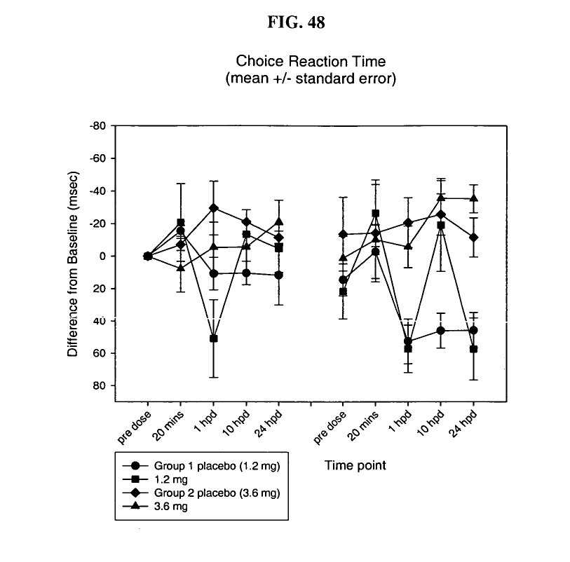

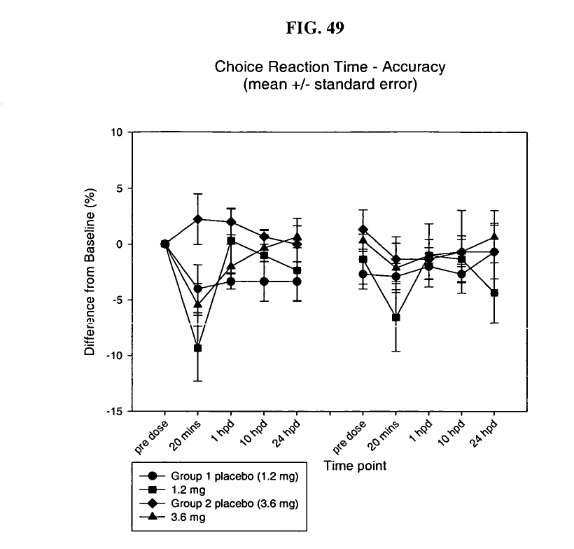

The data for Choice Reaction Time (FIG. 48-49) showed only some fluctuation in performance over the study for the placebo groups and the 3.6 mg dose, with the 1.2 mg dose showing much greater fluctuation and much larger standard error bars. Several large ‘peak’ declines were seen with 1.2 mg, most notably at 1 hour on Day 1 and 1 and 24 hours on Day 12, with some improvements at other time points. These declines were primarily due to 2 subjects.

The primary analysis showed a significant dose*time interaction (p<0.01), possibly due to the extreme reaction times obtained by two subjects. The comparisons showed a significant decrement for 1.2 mg against placebo at 1 hour (p<0.01), and a significant benefit for 3.6 mg against placebo at 24 hours (p<0.05). The secondary analysis showed no significant effect of dose or dose*day interaction.

Digit Vigilance

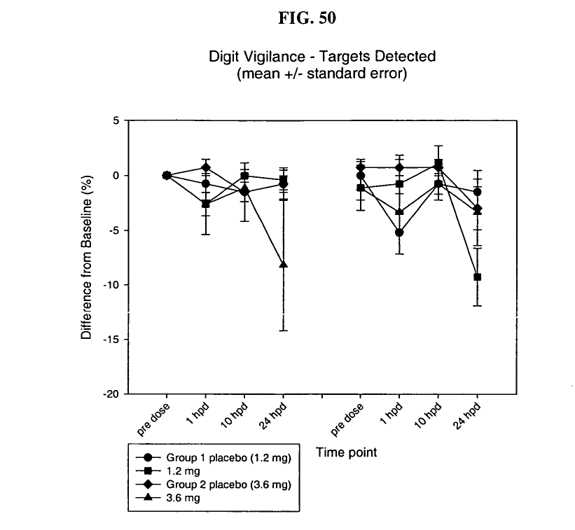

The data for Digit Vigilance Targets Detected (FIG. 50) showed some fluctuation in performance over the study, with declines for 3.6 mg at 24 hours on Day 1 (though with a marked increase in error) and for 1.2 mg at 24 hours on Day 12.

The primary analysis showed a significant dose*day*time interaction (p<0.05). The interaction resulted from the decline with 3.6 mg at 24 hours on Day 1, due to a single large decline for one subject, and a more general decline with 1.2 mg at 24 hours on Day 12. The secondary analysis showed a signal for a main effect of dose only (p<0.1). For placebo, baseline scores on the final day of dosing (99.3%) were slightly greater than Day 1 baseline assessment (98.9%). 1.2 mg showed slightly poorer scores at the final dosing day baseline assessment (95.6%) than Day 1 baseline assessment (96.7%), whilst 3.6 mg also showed slightly poorer scores at the final dosing day baseline assessment (97.4%) than Day 1 baseline assessment (98.5%).

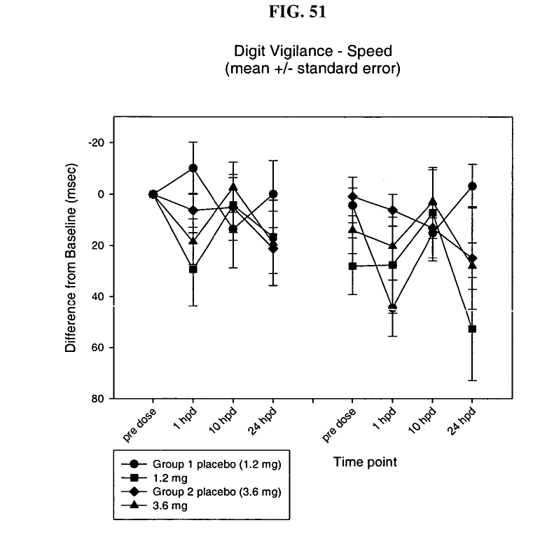

The data for Digit Vigilance Speed (FIG. 51) showed some fluctuation in performance over the study, but no clear pattern emerged. The primary analysis showed no significant effect of dose, or interaction between dose and day and/or time. The secondary analysis showed no significant effect of dose or dose*day interaction. The data for Digit Vigilance False Alarms showed small fluctuations in performance over the study, but did not indicate any clear dose or time based pattern.

Cognitive Working Memory Tasks

Numeric Working Memory

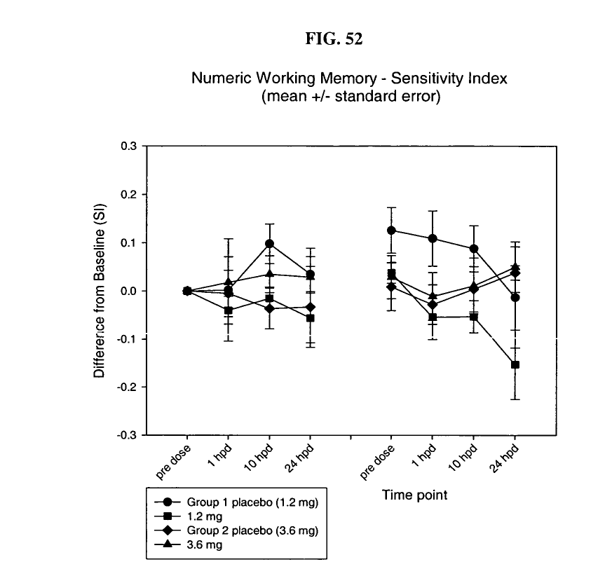

The data for Numeric Working Memory Sensitivity Index (FIG. 52) showed some fluctuation in performance over the study, with generally overlapping error bars. Some variation was seen between groups and doses in Day 1 pre-dose (baseline) performance, which was equal to later variation in group/dose performance. The primary analysis showed no significant effect of dose, or interaction between dose and day and/or time. The secondary analysis showed no significant effect of dose or dose*day interaction.

Numeric Working Memory Speed

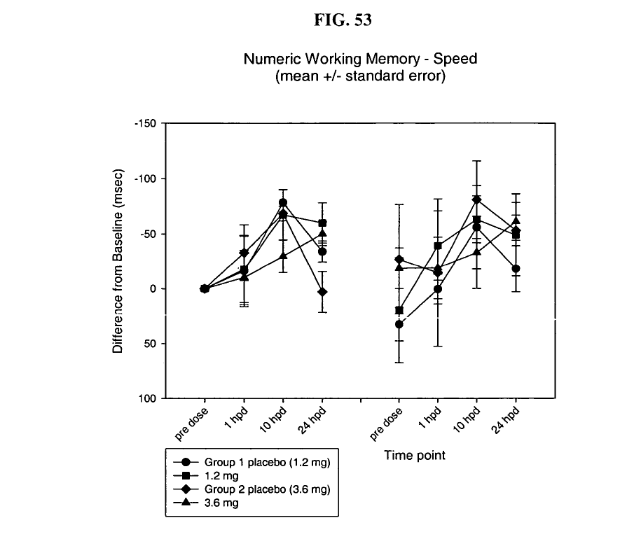

The data for Numeric Working Memory Speed (FIG. 53) showed some fluctuation in performance over the study, with generally overlapping error bars. The primary analysis showed no significant effect of dose, or interaction between dose and day and/or time. The secondary analysis showed no significant effect of dose or dose*day interaction.

Spatial Working Memory

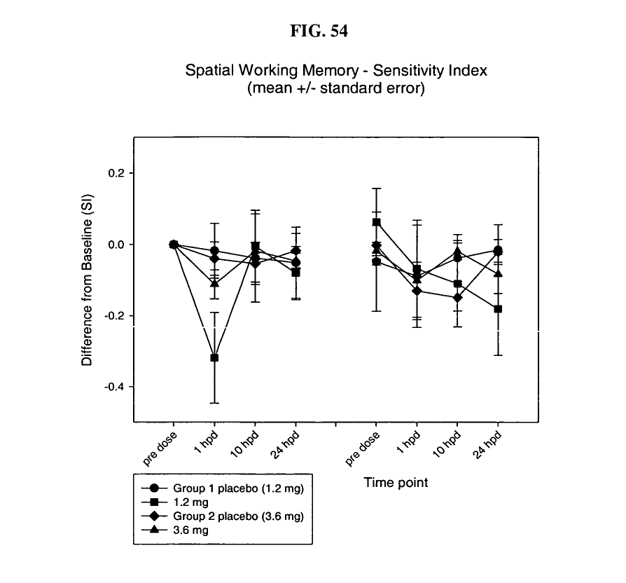

The data for Spatial Working Memory Sensitivity Index (FIG. 54) showed some fluctuation in performance over the study, with generally overlapping error bars for the 3.6 mg dose and placebo. A single large decline was seen for 1.2 mg at 1 hour on Day 1, and a small, increasing decline over the course of Day 12. The decline on Day 1 was largely due to extreme scores for two subjects and large error bars were also evident at 24 hours on Day 12. The primary analysis showed a significant main effect of dose (p<0.01). The comparisons only showed one significant decrement for 1.2 mg against placebo (p<0.05), and the significant main effect is therefore partly attributable to the extreme scores indicated above. The secondary analysis showed no significant effect of dose or dose*day interaction.

Spatial Working Memory Speed

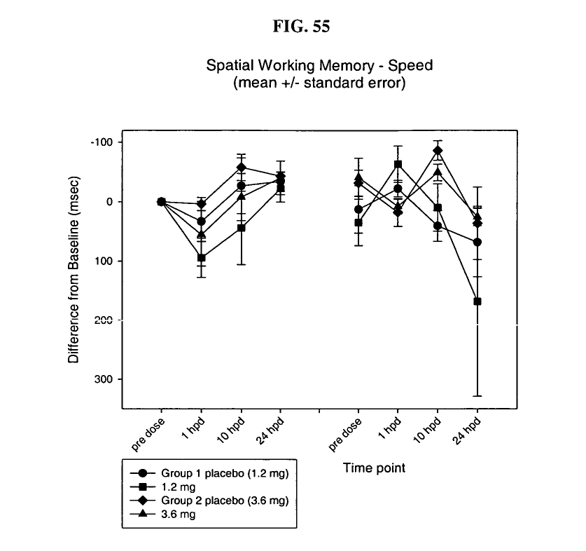

The data for Spatial Working Memory Speed (FIG. 55) showed some fluctuation in performance over the study, with generally overlapping error bars. There was some indication of a 1 hour decline for both active doses, most notably 1.2 mg on Day 1. A further large decline was seen for 1.2 mg at 24 hours on Day 19, though this was largely due to an extreme 1818 msec reaction time for a single subject (10935), who also had a 1058 msec reaction time at 1 hour on Day 1. The primary analysis showed no significant effect of dose, or interaction between dose and day and/or time. The secondary analysis showed no significant effect of dose or dose*day interaction.

Cognitive Episodic Secondary Memory Tasks

Immediate Word Recall

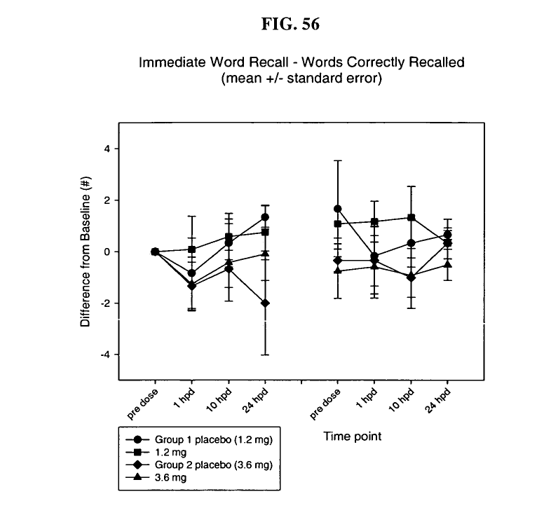

The data for Immediate Word Recall Words Correctly Recalled (FIG. 56) showed only small fluctuations in performance over the study, with generally overlapping error bars. The primary analysis showed no significant effect of dose, or interaction between dose and day and/or time. The secondary analysis showed no significant effect of dose or dose*day interaction. The data for intrusions and errors did not add to the interpretation of the task.

Delayed Word Recall

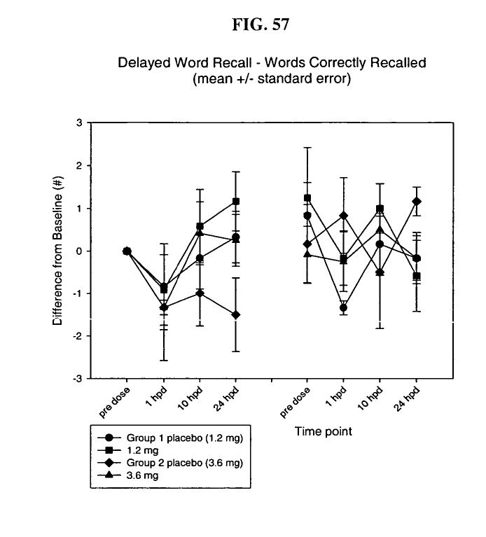

The data for Delayed Word Recall Words Correctly Recalled (FIG. 57) showed some fluctuation in performance over the study, with generally overlapping error bars, and little indication of a clear dose related pattern. The primary analysis showed no significant effect of dose, or interaction between dose and day and/or time. The secondary analysis showed no significant effect of dose or dose*day interaction. The data for intrusions and errors did not add to the interpretation of the task.

Word Recognition

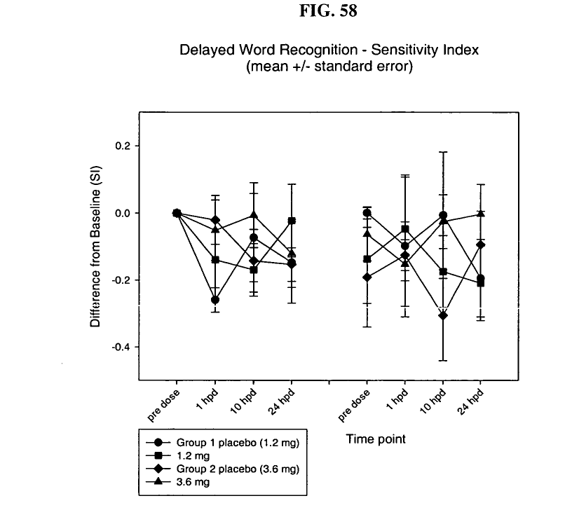

The data for Delayed Word Recognition Sensitivity Index (FIG. 58) showed some fluctuation in performance over the study, with generally overlapping error bars, and little clear indication of separation between the active doses and placebo. The primary analysis showed no significant effect of dose, or interaction between dose and day and/or time. The secondary analysis showed no significant effect of dose or dose*day interaction.

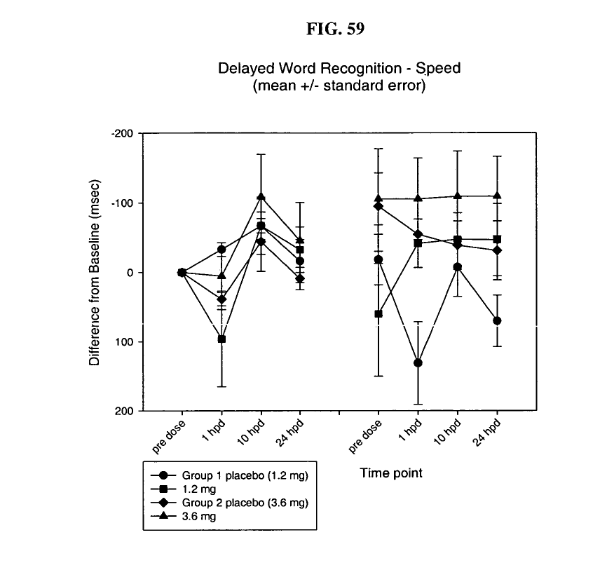

The data for Delayed Word Recognition Speed (FIG. 59) showed some fluctuation in performance over the study, with generally overlapping error bars. There was some indication of a decline for placebo at 1 hour on Day 12, though this was largely due to an extreme 1046 msec reaction time for a single subject. It should be noted that one subject, dosed with 1.2 mg, also had 3 reaction times greater than 1000 msec on this task measure, though this had less impact on group means. The primary analysis showed a significant dose*day interaction (p<0.05). The comparisons supported benefits for both 1.2 mg (p<0.05) and 3.6 mg (p<0.1) against placebo on the final day of dosing. The secondary analysis showed no significant effect of dose or dose*day interaction.

Picture Recognition

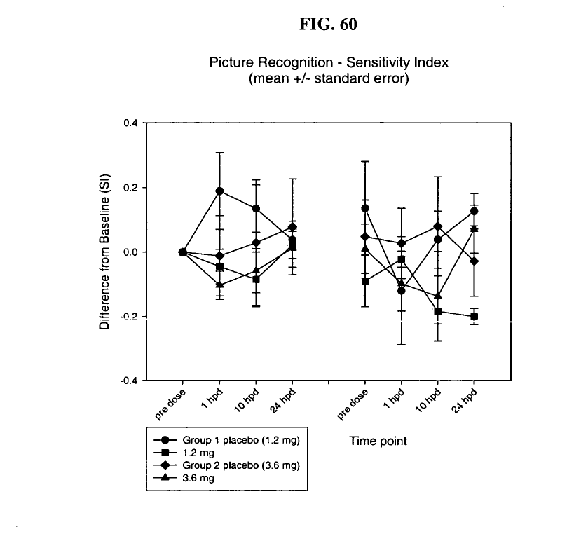

The data for Picture Recognition Sensitivity Index (FIG. 60) showed some fluctuation in performance over the study, with generally overlapping error bars, and little clear indication of separation between the active doses and the matched placebo group. However, some indication was seen for improvements for placebo on Day 1, particularly at 1 hour, and declines on Day 12, particularly at 1 hour. The primary analysis showed no significant effect of dose, or interaction between dose and day and/or time. The secondary analysis showed no significant effect of dose or dose*day interaction.

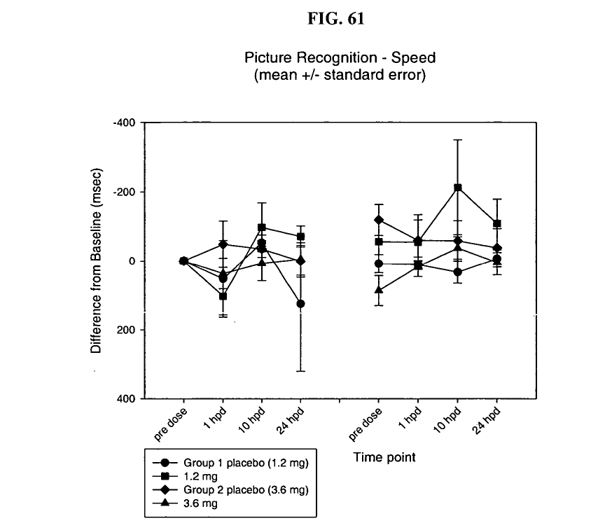

The data for Picture Recognition Speed (FIG. 61) showed some fluctuation in performance over the study, with generally overlapping error bars. Most notably a clear improvement in performance was seen for 1.2 mg at 10 hours on Day 12, whilst a decline was also seen at 1 hour on Day 1 for this dose. Further, there was some indication of a decline for 3.6 mg at pre-dose on Day 19, whilst placebo improved slightly. The primary analysis showed no dose*time interaction (p>0.05). The secondary analysis also showed no dose*day interaction (p>0.05).

Cognitive Motor Control Task

Tracking Task

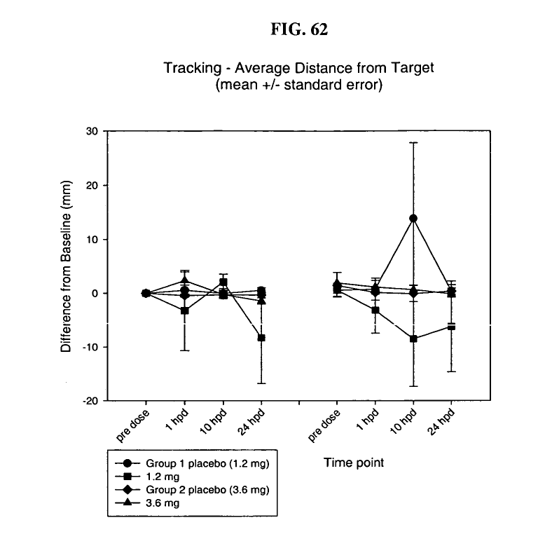

The data for Tracking Average Distance from Target (FIG. 62) generally showed little fluctuation in performance over the study, with overlapping error bars. However, particularly poor performance was seen for 1.2 mg at pre-dose and 10 hours on Day 1 and at pre-dose on Day 12, and for placebo at 10 hours on Day 12, all with large error bars. These group means, at each time, were associated with extremely poor performance (>60 mm) for two subjects (1 placebo; 11.2 mg). The primary analysis showed no significant effect of dose, or interaction between dose and day and/or time. The secondary analysis showed no significant effect of dose or dose*day interaction.

Subjective Drug Rating

In Group I, a placebo response was observed on Day 1 as indicated by a persistent “feel the drug”, “liking”, and some slight “disliking” scores that persisted through 4 hours after dosing. On Day 12, the scores were diminished with placebo but did not reach a score of zero. The six subjects receiving 1.2 mg of delta-9-THC on Day 1 reported “feel the drug” and “liking” scores that were slightly larger than those who received placebo. The greatest scores from subjects receiving active drug in Group I were associated with the “disliking” item, indicating a degree of drug-induced adverse effects. On Day 12, the “feel the drug” and “liking” scores were diminished; however, the “disliking” scores were approximately the same or slightly higher.

In Group II, a slight placebo response was observed on Day 1 which had diminished by Day 19. The effects of 3.6 mg of delta-9-THC MDI on Day 1 were associated with significantly larger “feel the drug”, “liking”, and “disliking” scores than the placebo on Day 1. On Day 19, the scores following active drug were less than the scores observed on Day 1 after active treatment, but were still much larger than the placebo scores.

Addiction Research Center Inventory (ARCI)-49 Data

The data for Group I and Group II were pooled and submitted to a mixed effects general linear model analysis. Two sets of analyses were done. The first was for the pre-drug conditions. The fixed effect of day and the fixed effect of condition were non-significant for all of the five scale scores, indicating similarity of pre-drug baseline score. A second analysis compared condition, the first dosing day, the last dosing day, the four visits including pre-drug, and the subjects in the analysis of variance.

With regard to the Morphine Benzedrine Group (MBG), all factors except for visit were non-significant, including the fixed effect factors of condition and day. The significant effect for visit suggests that there was some effect over time for the MBG scale scores. Examination of the least squares means did not show a significant decrease for the 10 hour post-dose observation. Comparison of mean scores suggests that the 3.6 mg dose produced some MBG effect at 1 hour post-dose that was diminished after repeated administration, as indicated by the lower MBG scale scores on the last dose day.

The analyses for the Lysergic Acid Diethylamine (LSD) scale indicated no significance for condition, day, visit, or any of the interaction terms.

For the Pentobarbital-Chlorpromazine-Alcohol (PCAG) scale, the fixed effects of condition and day were non-significant; however, visit and the interaction term of condition times visit were highly significant at less than p=0.001. Examination of the least squares means indicated that this was an effect at 1 hour post-dose on Day 1. The 1 hour post-dose PCAG scores on the last dosing day were greater than the Day 1 observations.

For the Benzedrine Group scale, no significant fixed effects were seen for condition or day or the condition by day. Interaction terms were, however, significant for the visit and the condition by visit interaction terms. Examination of the least squares means indicated these were effects seen at 1 hour post-dose, and there appeared to be a significant decrease in effects.

The Amphetamine Scale (AS) showed no significant effects for condition. There was, however, a significant fixed effect for day. None of the interaction terms were significant. From examination of the least squares means, there appeared to be a significantly lesser effect on the second dosing day rather than the first dosing day.

Pharmacokinetic/Pharmacodynamic Relationship

The pharmacodynamic effect of delta-9-THC on heart rate showed a maximum effect within 2-5 minutes after single dosing, and a duration of 2-4 hours for both dose levels. Both the duration and the maximum effect coincided with delta-9-THC but not 11-OH-THC maximum plasma concentrations. The delayed increase in 11-OH-THC plasma concentrations between 2 hours and 4 hours post-dose was not associated with a clear effect on heart rate.

A pharmacodynamic effect on blood vessels in the eyes was absent in this study, except one report of slight conjunctiva congestion in one subject receiving 1.2 mg delta-9-THC. This effect was observed from 10 minutes up to and including 3 hours after dosing on Day 12, and therefore coincided with delta-9-THC and 11-OH-THC maximum plasma concentrations.

Subjective Drug Rating (SDR) Questions 1 and 2 produced the largest scores at time-points clearly later than tₘₐₓ for delta-9-THC but close to tₘₐₓ for 11-OH-THC, suggesting that 11-OH-THC may be involved in these responses. Duration of the SDR responses was also prolonged, especially compared to effects on heart rate. SDR Question 3 produced marked scores immediately (5 minutes) after dosing on Day 1, which coincided with maximum plasma concentrations of delta-9-THC. Responses were observed over a period ranging between 2 and 24 hours after dosing and tended to be associated with both delta-9-THC and 11-OH-THC plasma profiles.

Safety

Adverse Events

A brief summary of adverse events (AEs) is presented in Table 9. A total of 58 AEs were reported in 17 out of 18 subjects (94.4%). There were two pre-treatment AEs (in Group II) and 56 TEAEs (defined as all AEs that began or worsened after the subject received the first dose of study medication until the subject was released from the unit). Twenty-six TEAEs were reported in nine subjects (100%) in Group I and 30 TEAEs in eight subjects (88.9%) in Group II. Among subjects receiving placebo treatment, six events were reported by three subjects in Group I, and four events by two subjects in Group II. Among subjects treated with active drug, 27 events were reported by all six subjects in Group I, and 19 events by all six subjects in Group II. There were five events in two subjects on placebo and 13 events in all six subjects on active drug in Group I and 17 events in all six subjects on active drug in Group II that were considered probably or possibly related to study drug. There were no serious or severe AEs, and no AEs leading to premature termination.

TABLE 9
Brief summary of adverse events

| | | Group I | | |
|---|---|---|---|---|
| | All groups | All group I | placebo | 1.2 mg |
| Total subjects at risk | 18 | 9 | 3 | 6 |
| Subjects without any TEAE | 1 (5.6%) | | | |
| Subjects with at least one AE | 17 (94.4%) 58 | 9 (100%) 26 | 3 (100%) 6 | 6 (100%) 20 |
| Any serious TEAE | 0 (0.0%) 0 | | | |
| Any TEAE leading to | 0 (0.0%) 0 | | | |
| premature termination | | | | |
| Any severe TEAE | 0 (0.0%) 0 | | | |
| Any related TEAE | 14 (77.8%) 35 | 7 (77.8%) 16 | 1 (33.3%) 3 | 6 (100%) 13 |
| Any serious AE | 0 (0.0%) 0 | | | |
| | | Group II | | |
| | | All group II | placebo | 3.6 mg |
| Total subjects at risk | 18 | 9 | 3 | 6 |
| Subjects without any TEAE | 1 (5.6%) | 1 (11.1%) | | |
| Subjects with at least one AE | 17 (94.4%) 58 | 8 (88.9%) 32 | 2 (66.7%) 5 | 6 (100%) 27 |
| Any serious TEAE | 0 (0.0%) 0 | | | |
| Any TEAE leading to | 0 (0.0%) 0 | | | |
| premature termination | | | | |
| Any severe TEAE | 0 (0.0%) 0 | | | |
| Any related TEAE | 14 (77.8%) 35 | 7 (77.8%) 19 | 1 (33.3%) 2 | 6 (100%) 17 |
| Any serious AE | 0 (0.0%) 0 | | | |

n (x %) z: n = number of subjects, x = percentage of subjects receiving treatment, z = number AEs

TEAEs listed by relationship to study drug are summarized in Table 10 below; as there were no adverse events other than mild, there is no table of TEAEs by severity.

TABLE 10
Summary of TEAEs by relationship

| | |
|---|---|
| Probable/ | Unlikely/ |
| Possible | Unrelated |

| | E | N | E | N |
|---|---|---|---|---|
| Group I | 16 | 7 | 10 | 5 |
| Placebo | 3 | 1 | 3 | 2 |
| Active | 13 | 6 | 7 | 3 |
| Group II | 19 | 7 | 11 | 4 |
| Placebo | 2 | 1 | 2 | 1 |
| Active | 17 | 6 | 9 | 3 |

E = number of adverse events,
N = number of subjects; all of mild intensity

In total, 56 TEAEs occurred in 17 subjects, of which 26 TEAEs occurred in Group I (nine subjects, 100%) and 30 TEAEs occurred in Group II (eight out of nine subjects, 88.9%). Of the 56 TEAEs, 35 (16 in Group I and 19 in Group II) were considered probably or possibly related to the study drug. All TEAEs were of mild intensity.

The most frequently reported TEAEs were cough (14 events in 13 subjects, seven events in seven subjects in Group I, seven events in six subjects in Group II, all probably or possibly related to the study drug), somnolence (four events in four subjects in Group I, two events in two subjects in Group II, 2 events in two subjects in each group probably or possibly related) and headache (three events in three subjects in each group). Among probably or possibly related TEAEs, euphoric mood showed one event in one subject in Group I and three events in three subjects in Group II.

Most TEAEs were transient and had resolved without sequelae by the follow-up visit. Liver enzymes were elevated at follow-up, however, in three subjects in Group II, all receiving 3.6 mg delta-9-THC. Among them, two subjects were lost to further follow-up and outcome was documented as unknown. In the case of the other subject, all tests were repeated 6.5 weeks after the follow-up visit and were found to have returned to the normal range.

Analysis of Adverse Events

A total of 10 TEAEs were reported in five out of six subjects receiving placebo treatment, all three in Group I (100%) and two out of three in Group II (66.7%), among whom only two subjects (one in each group, 33.3%) reported TEAEs that were considered to be probably or possibly related to study drug. All subjects receiving active treatment reported TEAEs that were probably or possibly related to study drug.

Among placebo-treated subjects, headache and cough were the most frequent TEAEs (three events each, headache in three subjects, cough in two), with headache being assessed as unlikely or unrelated to study drug and cough as probably or possibly related to study drug on all occasions. Among subjects receiving 1.2 mg delta-9-THC in Group I and 3.6 mg delta-9-THC in Group II, all six in Group I (100%) and five out of six in Group II (83.3%) reported cough during most of the dosing period. This event was the most frequent TEAE and was considered probably related on all occasions. Somnolence, either of brief or prolonged duration (1 hour-8 days), was reported four times, by two subjects receiving active treatment in each group (33.3%) and was considered possibly or probably related on all occasions; euphoric mood (of 1- to 7.5-hours duration) was reported four times as well, once by one subject in Group 1 (16.7%) and once by three subjects in Group III (50.0%), again all considered possibly or probably related.

Cough was almost exclusively reported as being intermittent in subjects on active treatment, with duration between four and nine days in Group I and between six and 18 days in Group II. It remains unclear however whether coughing occurred exclusively during and immediately after administration of study drug, or whether there was also coughing during longer time frames after dosing. Cough was reported as intermittent, with a duration of 5.5 days, in one subject receiving placebo, and was only very brief (1 minute duration) on two dosing occasions in another subject receiving placebo. Thus, cough was primarily associated with inhalation of the active drug rather than the vehicle.

Under delta-9-THC treatment, a total of four complaints related to the eyes were reported by three subjects. One subject, receiving 1.2 mg delta-9-THC, reported irritation from Day 6 onwards and burning eyes briefly on Day 12. Another subject, receiving placebo in Group II, reported eye pain briefly on Day 1, and another subject (who received 3.6 mg delta-9-THC) reported pressure on the eyes briefly on Day 1. Headache and fatigue were both reported three times, by a total of three subjects receiving active treatment (one subject in Group I, two in Group II); the relationship to study drug was considered possibly (one fatigue and one headache in one subject in Group II) or unlikely related in all other cases.

There were also four occurrences of elevated liver function tests, in three subjects in Group II. Two of these, elevated transaminases were considered possibly related to study drug. One subject showed elevated ASAT as well as total bilirubin, lactate dehydrogenase (LDH) and creatine kinase (CK) levels and various urinary analytes, all regarded as unlikely related to the study drug and without clinical signs or symptoms.

12-Lead ECG

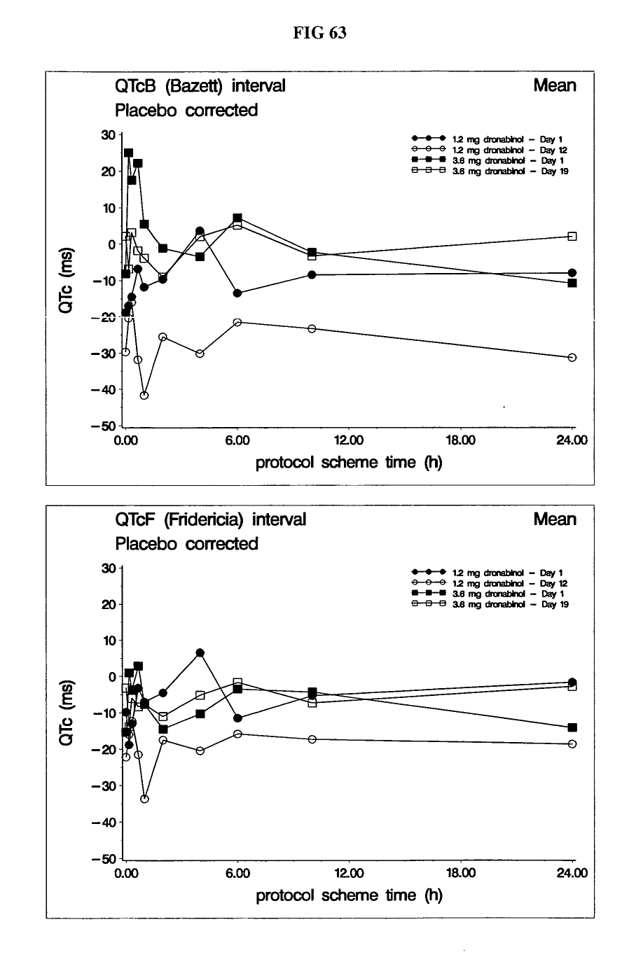

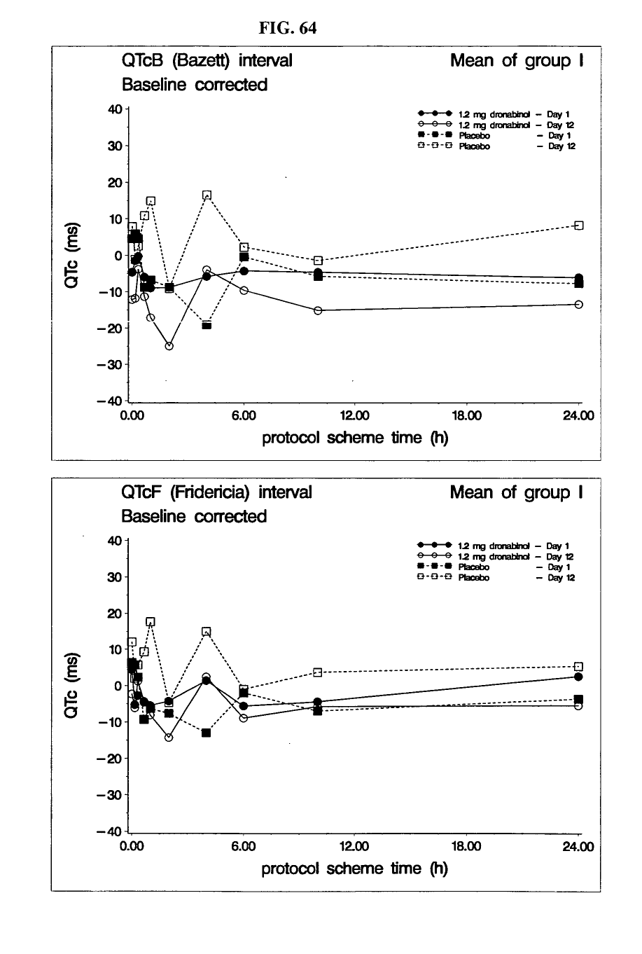

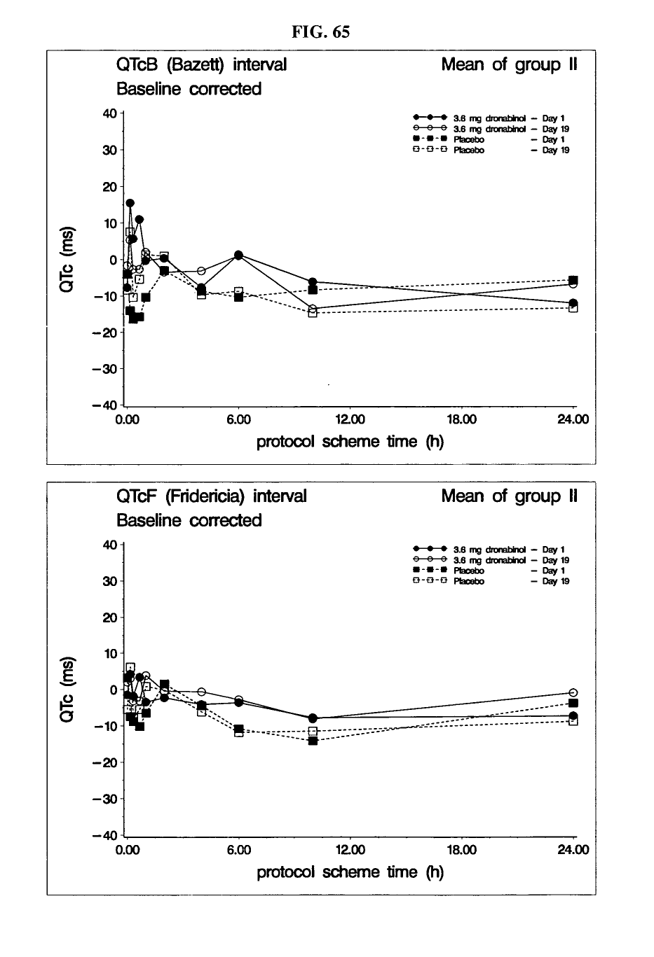

Graphs of placebo-corrected QTc-intervals (Bazett's and Frederica's) are shown in FIG. 63 and baseline-corrected QTc-intervals (Bazett's and Fredericas) for Group I are shown in FIG. 64 and for Group II are shown in FIG. 65

A number of individual abnormalities were observed, but ECG parameters showed no clinically significant abnormalities or trends during single and multiple inhalational dosing with delta-9-THC.

As a possible exception to the lack of any trends, QTc-intervals according to Bazett were slightly prolonged (by an average 10 to 20 msec) immediately after the first dose in Group II; QTc returned towards baseline by 1 hour after dosing. Such effect was not observed in subjects receiving placebo in Group II, nor in subjects in Group I or in Group II on Day 19. Individual QTc values were less than 450 msec at each assessment time point, with mean values <395 msec and individual values <422 msec.

The slight and apparent increase in QTc based on the Bazett correction most likely resulted from an overcorrection as a result of the drug-induced increase in heart rate, as it is known that Bazett's QTc correction should be interpreted with caution for compounds that cause tachycardia. Thus, QTc-intervals as calculated using Fridericia's correction showed no apparent effect of delta-9-THC on QTc whatsoever.

As also discussed above, heart rate increased compared to baseline immediately after dosing on Day 1 in both groups, reaching a mean value of 67 bpm in Group I (mean increase above baseline: 4 bpm, mean increase above pre-dose value on Day 1: 13 bpm) and of 81 bpm in Group II (mean increase above baseline: 14 bpm, mean increase above pre-dose value on Day 1: 21 bpm), at 20 minutes after dosing. Heart rate returned to baseline in the course of the 40 minutes (Group I) or 4 hours (Group II) after dosing. The effect on heart rate was absent in placebo-treated subjects on Day 1, and of similar, limited magnitude during placebo and active treatment on Days 12 (Group I) and 19 (Group II). This may be taken to suggest that this was a drug-induced effect that leveled off in the course of multiple dosing.

Telemetric Cardiac Monitoring

There were no clinically significant abnormalities observed during telemetric cardiac monitoring. One minor individual abnormality was reported for Subject 10942, who showed two events of ventricular ectopic beats on the first day of dosing, rated as abnormal but not clinically significant.

Pulmonary Function

All subjects produced normal lung function results (FEV1 and FVC), both at pre-study and follow-up visits and at all time-points during the study, except two. These two subjects produced normal FVC at all times during the study and borderline normal FEV1 (80% of predicted) at pre-study screening, however FEV1 was below normal (67 and 70% of predicted, not clinically significant) in two measurements pre-dose on Day 1. One subject was enrolled to receive 1.2 mg delta-9-THC after mutual agreement between Sponsor and Medical Investigator. FEV1 remained essentially stable during the study (lowest result 63% on Day 11, after six days of dosing with 1.2 mg delta-9-THC), however at the follow-up visit FEV1 was 58% of predicted, which was regarded as an abnormal, not clinically significant observation due to slight airway obstruction. This was not followed up further.

Summary

Pulmonary delivery of delta-9-THC provided rapid systemic absorption both after single and multiple doses of 1.2 mg and 3.6 mg. A dose-related increase in Cₘₐₓ and AUC was observed both after single and multiple dose administration. Terminal elimination half-lives were estimated to be approximately 93 hour for delta-9-THC, 40 hours for 11-OH-THC, and 30 hour for THC-COOH.

Heart rate increased in a dose-dependent fashion after single dose inhaled delta-9-THC administration. Heart rate effects were similar to placebo after 1-2 weeks of multiple dosing with inhaled delta-9-THC.

Minimal cognitive function effects were observed after multiple dose administration of inhaled delta-9-THC at dose levels of 1.2 mg and 3.6 mg.

Conjunctiva congestion was not clinically significant at the dose levels studied. An observation of slight intensity was noted in a single subject.

A total of 56 TEAEs occurred in 17 subjects; 26 occurred in all nine subjects in Group I, and 30 in eight out of nine subjects in Group II. All TEAEs were of mild intensity and resolved spontaneously and without sequelae. Thirty-five (35) TEAEs were considered possibly or probably related to the study drug.

In Group I (1.2 mg dose level), the most frequently reported TEAEs were cough and somnolence. In Group II (3.6 mg dose level), the most frequently reported TEAEs were cough, headache, and euphoric mood.

Increased liver function tests were observed in three out of nine subjects receiving 3.6 mg delta-9-THC for two weeks, which was considered possibly related in two of the three subjects.

No dose-related trends or clinically significant changes were found in the vital signs, ECG, physical examination, telemetric monitoring and pulmonary function tests.

No deaths or serious adverse events occurred throughout the study.

Pulmonary inhaled delta-9-THC was considered safe and well-tolerated after single and multiple dosing with 1.2 and 3.6 mg.

Test Methods

Pharmacokinetic

Whole blood samples were collected according to the assessment flowchart in EDTA tubes through venepuncture or indwelling catheter. Samples were immediately placed on ice and subsequently centrifuged within 30 minutes at 1,500 g for 10 minutes. Plasma was transferred into a 5 ml screw cap polypropylene tube, stoppered and stored at below minus 20° C. until transfer to the analytical laboratory for analysis. Delta-9-THC, 11-OH-THC (active metabolite), THC-COOH (inactive metabolite) were extracted from the plasma using solid phase extraction followed by quantification using Turbo ionspray LC-MS/MS. The measurement ranges were 0.05-30 ng/ml for delta-9-THC, 0.04-30 ng/ml for 11-OH-THC, and 0.25-10 ng/ml for THC-COOH.

These plasma concentrations were used to calculate the AUC (area under the plasma concentration-time curve from time zero extrapolated to infinity), AUC₀₋ₜ (area under the plasma concentration-time curve from time zero to the last quantifiable concentration), Cₘₐₓ (maximal plasma concentration), l_{z} (terminal first order elimination rate constant), t_{1/2} (elimination half-life, the time required for the drug plasma concentration to decrease by 50%), tₘₐₓ (time at which the maximal plasma concentration was observed).

Number of subjects, mean, standard deviation, coefficient variable (%) and geometric mean were calculated for all pharmacokinetic parameters.

For Example 1: A mixed ANOVA model with group and dose fixed effect and subject within group by dose as random effect was performed on the logarithms of the dose normalized pharmacokinetic parameters AUC, AUC₀₋ₜ and Cₘₐₓ. The overall treatment effect was tested by conventional F-test with Satterthwaites correction. Dose proportionality was tested using Helmert Contrasts and Reverse Helmert Contrasts. The within and between subject coefficient of variances were calculated from the estimated covariance parameters. Per dose level geometric means with 90% confidence interval were calculated for the dose-normalized values from the least squared means analysis outcomes. The difference between young and elderly subjects was explored using a one-way ANOVA.

For Example 2: The statistical analysis encompassed an exploratory analysis of the single and multiple dose pharmacokinetics and dose proportionality of two dose levels of inhaled delta-9-THC in healthy subjects. Descriptive statistics included number of subjects, mean, standard deviation, coefficient of variation (%) and geometric mean for all pharmacokinetic parameters.

The exploratory analysis was performed on the logarithms of the dose-normalized pharmacokinetic parameters AUC_{(0-inf)}, AUC₍₀₋ₜ₎, AUC_{(0-τ)} and Cₘₐₓ. Dose proportionality was tested both at single dose and at multiple dose (steady state) by comparing the dose groups for all pharmacokinetic parameters by an analysis of variance with dose as fixed effect.

Per dose level the least squares means and standard errors of the log-dose-normalized parameters, and after anti-logarithmic transformation the geometric means and 90% C.I. was given.

Dose proportionality was tested by the contrast between the dose groups. For this contrast on the logarithmic scale, p-values were given. After anti-logarithmic transformation, the resultant ratios of geometric means and 90% confidence intervals were given.

The between subject coefficient of variation (CV) was calculated from the estimated covariance parameters.

Accumulation was tested by comparing the steady state values for AUC_{(0-τ)} with the single dose values for AUC_{(0-τ)} by a mixed model analysis of variance with dose and day (with levels single dose and steady state) as fixed effects and subject within dose as random effect.

Linearity was tested by comparing the steady state values of AUC_{(0-τ)} with the single dose values of AUC₍₀₋ᵢₙₜ₎ by a mixed model analysis of variance with dose and day (with levels single dose and steady state) as fixed effects and subject within dose as random effect.

Linearity was tested by the contrast between the days (steady state versus single dose). For this contrast on the logarithmic scale, p-values were given. After anti-logarithmic transformation, the resultant ratios of geometric means and 90% confidence intervals were given.

Pharmacodynamic Measurements

For Examples 1 and 2: Heart rate was obtained from the blood pressure recordings or using telemetry.

Conjunctiva Congestion was rated by a trained observer on a scale of 0-3 based on the blood vessels present as follows:

0—None (The white of the eye was not affected)

1—Slight (A part of the eye shows a number of blood vessels)

2—Moderate (The complete white of the eye shows a network of bigger and smaller blood vessels without complaints)

3—Considerable (The compete white of the eye is red caused by a pattern of bigger vessels. This is associated with complaints.)

Absolute values of heart rate and conjunctive congestion scores were used to calculate changes from baseline.

The Bond-Lader Visual Analogue Scale was used to measure subjective changes in mood and alertness after drug administration. Sixteen horizontal, visual analogue scales were used, with the subject required to make a clear mark across each line. The sixteen questions represented opposing terms that assessed temperament such as: Alert-drowsy, calm-excited, happy-sad, mentally slow-quick-witted, lethargic-energetic.

Cognitive Assessment to assess attention/working memory, perceptual/motor, abstraction/executive, simple reaction time, learning and verbal domains were performed. The full 25 minute Cognitive Drug Research battery (25 minutes) was administered pre-dose, and at 1, 5 and 24 hours post dose. A shortened battery (5 minutes) was administered 20 minutes post-dose to provide data on early effects of the compound. Tasks were computer controlled with answers using two buttons “Yes” or “No”. For the tracking task, a joy stick was also used. For the word recall tasks, subjects wrote the words down on paper. The following tests were administered:

Immediate Word Recall: Fifteen words were presented on screen at a rate of 1 every 2 seconds for the subject to remember. One minute was given to recall as many words as possible.

Simple Reaction Time: Each subject was instructed to press the “Yes” response as quickly as possible every time “Yes” was displayed on the screen. Fifty stimuli were presented with varying inter-stimulus intervals.

Digit Vigilance: A target digit was randomly selected and constantly displayed on the right hand side of the screen. A series of digits was then presented in the center of the screen at 150/minute over 3 minutes. The subject was required to press “Yes” as quickly as possible every time the digit in the series matched the digit on the screen. Forty-five targets were in the series.

Choice Reaction Time: Either “No” or “Yes” was presented on the screen and the subject pressed the corresponding button as quickly as possible. There were 50 trials with each word selected randomly with equal probability and varying inter-stimulus intervals.

Tracking: The subject used a joystick to track a randomly moving target on the screen for one minute. The distance off per target was recorded.

Spatial Working Memory: A picture of a house was presented on the screen with four of its nine windows lit. The subject memorized the position of the lit windows. For each of 36 subsequent presentations, the subject was required to decide whether the one window that was lit was also lit in the original presentation using the buttons.

Numeric Working Memory: A series of five digits was presented to the subject to hold in memory. This was followed by 30 probe digits for each of which the subject had to determine whether it was in the original series using the buttons as quickly as possible. The test was repeated twice with different series and probes.

Delayed Word Recall: The subject was given 1 minute to recall as many of the words as possible.

Word Recognition: The original plus 15 distractor words were presented one at a time randomly. The subject had to indicate whether he or she recognized each as being from the original list.

Picture Recognition: The original plus 20 distractor pictures were presented one at a time randomly. The subject indicated whether he or she recognized each as being from the original series.

Subjective Drug Ratings

The subject responded to each of three questions on a scale of 0 to 100 with 0 meaning no effect/not at all and 100 meaning maximum/very much:

How much of a drug effect or high do you feel?

How much do you like the drug?

How much do you dislike the drug?

Addiction Research Center Inventory (ARCI, shortened version)

The ARCI is a true/false questionnaire developed to specifically measure the subjective effects of drugs which have diverse pharmacological actions. The phenobarbital-chlorpromazine-alcohol (PCAG), morphine-benzedrine, and lysergic acid diethylamide subgroup scales were used to assess sedation, euphoria, and dysphoria. Questions from the marijuana subscale were also included.

Subjective drug ratings and cognitive measurement scores were presented as absolute values only with separate tables summarizing gender difference and age differences.

For Example 1: All pharmacodynamic variables were evaluated using descriptive statistics for all study evaluations.

For Example 2:

Conjunctiva Congestion, Heart Rate, and Subjective Ratings

Changes from baseline in conjunctiva congestion and heart rate were compared for all time-points measured, and summarized using descriptive statistics. Subjective ratings were individually tabulated and sorted by treatment group, subject number, and time, and summarized using descriptive statistics. Separate tables were provided to summarize differences between males and females if applicable. Pharmacodynamic assessments were compared with delta-9 THC and 11-OH-THC blood concentrations and summarized using descriptive statistics.

Cognitive Function, Bond-Lader VAS, and ARCI-49

Analysis of cognitive function data, VAS assessments, and the ARCI-49 questionnaire included the following:

Summary statistics (n, mean, sd, median, min, max) were calculated for each measure at each time by group and treatment. For each measure, ‘first day of dosing’ (Day 1) pre-dose data was subtracted from the data at each post-dosing time on that day, and ‘second day of dosing’ (Day 12 or Day 19) pre-dose data was subtracted from the data at each post-dosing time on that day, to derive ‘difference from baseline’ scores. Figures (mean±standard error) were plotted over time using the unadjusted scores and derived ‘difference from baseline’ scores.

Primary Analysis—Repeated measures ANCOVA was conducted on the difference from baseline data using SAS® PROC MIXED. Fixed terms were fitted to the model for dose, day, time, and the dose*time, dose*day, and dose*time*day interactions. A random effect of subjects was fitted to the model. Pre-dose (baseline) scores by Day were used as a covariate. Significance of the interactions was tested at the 0.05 level. All testing was two-tailed. If the interaction was found to be significant, appropriate comparisons were conducted between treatments. This analysis approach results in identical estimated treatment effects to an analysis of the raw outcome variables, analyzed with baseline as a covariate.

Secondary Analysis—ANOVA was conducted on the pre-dose data using SAS® PROC MIXED. Fixed terms were fitted to the model for dose, day, and the dose*day interaction. A random effect of subjects was fitted to the model. Significance of the interactions was tested at the 0.05 level. All testing was two-tailed. If the interaction was found to be significant, appropriate comparisons were conducted between treatments.

In both analyses a pooled placebo group was used.

Safety

Adverse events were recorded based on original descriptions from subject responses to queries. Complete medical histories and physical examinations were conducted. Vital signs were monitored, ECGs were recorded along with pulmonary function tests and laboratory examinations.

For Example 2:

Treatment-emergent adverse events (TEAEs), defined as any event that begins or worsens after treatment with study medication, were summarized by MedDRA system organ class (SOC) for each treatment group. The number and percentage of subjects with TEAEs was tabulated for each treatment and with respect to maximum severity and relationship to study medication.

Listings of values for each subject were presented with abnormal or out of range values for vital signs, laboratory assays, ECGs, pulmonary function tests, and physical examinations. Descriptive statistics (n, mean, standard deviation (SD), minimum, median, maximum) for all clinical laboratory safety parameters, ECGs, pulmonary function tests, and vital signs were provided. ECGs and physical examinations were to be summarized in shift tables to show changes from baseline between normal and abnormal findings.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

What is claimed is:

1. A method of administering delta-9-tetrahydrocannabinol to a patient in need thereof comprising the steps of: administering to said patient from a metered dose inhaler a pharmaceutical composition comprising delta-9-tetrahydrocannabinol, an alcohol and a propellant, wherein:
a) said delta-9-tetrahydrocannabinol is present in the composition in a concentration of about 0.1 mg/50 mcL to about 2.0 mg/50 mcL; and
b) said patient achieves a blood plasma concentration of delta-9-tetrahydrocannabinol of about 5 ng/mL to about 70 ng/mL within about 10 minutes of initiation of said administration.

2. The method of claim 1 wherein said blood plasma level concentration is achieved within about 5 minutes of initiation of said administration.

3. The method of claim 1 wherein said blood plasma level concentration is achieved within about 2 minutes of initiation of said administration.

4. The method of claim 1 wherein said administration from said metered dose inhaler delivers from about 1 mg to about 10 mg delta-9-tetrahydrocannabinol per actuation of the inhaler.

5. The method of claim 4 wherein said administration from said metered dose inhaler delivers from about 2 mg to about 8 mg delta-9-tetrahydrocannabinol per actuation of the inhaler.

6. The method of claim 4 wherein said administration from said metered dose inhaler delivers from about 3 mg to about 4 mg delta-9-tetrahydrocannabinol.

7. The method of claim 1 wherein the propellant is BFA 134a.

8. The method of claim 1 wherein the alcohol is ethanol.

9. The method of claim 1 wherein said patient is suffering from anorexia.

10. The method of claim 8 wherein said anorexia is a symptom of AIDS or HIV infection.

11. The method of claim 1 wherein said patient is suffering from nausea and/or vomiting.

12. The method of claim 10 wherein said nausea and vomiting is the result of cancer chemotherapy.

13. The method of claim 1 wherein said blood plasma concentration is between about 30 and about 60 ng delta-9-tetrahydrocannabinol/mL plasma.

14. The method of claim 1 wherein said delta-9-tetrahydrocannabinol in said metered dose inhaler is present in a concentration of about 0.3 mg/50 mcL of solution to about 1.5 mg/50 mcL of solution.

15. The method of claim 1 wherein said delta-9-tetrahydrocannabinol in said metered dose inhaler is present in a concentration of about 0.8 mg/50 mcL of solution to about 1.3 mg/50 mcL of solution.

16. The method of claim 1 wherein said delta-9-tetrahydrocannabinol is synthetic.

17. The method of claim 1 wherein said delta-9-tetrahydrocannabinol is natural.

18. The method of claim 1, wherein said patient is suffering from migraine headaches.

19. The method of claim 1, wherein said patient is suffering from multiple sclerosis.
